(19) United States
(12) Patent Application Publication
(10) Pub. No.: US 2005/0019784 A1
(43) Pub. Date: Jan. 27, 2005

(54) Method and apparatus for nucleic acid sequencing and identification

(76) Inventors: Xing Su, Cupertino, CA (US); Andrew A. Berlin, San Jose, CA (US)

Correspondence Address: LISA A. HAILE, Ph.D.;ATTORNEY FOR INTEL CORPORATION, GRAY CARY WARE & FREIDENRICH LLP, 4365 Executive Drive, Suite 1100, San Diego, CA, 92121-2133, US

(21) Appl. No.: 10/701,650

(22) Filed: Nov. 4, 2003

Related U.S. Application Data
(63) Continuation-in-part of application No. 10/153,125, filed on May 20, 2002.

(51) Int. Cl. C12Q 1/68; C12M 1/34

(52) U.S. Cl. 435/6; 435/287.2

(57) ABSTRACT

The methods and apparatus disclosed herein are of use for sequencing and/or identifying nucleic acids. Nucleic acids containing labeled nucleotides may be synthesized and passed through nanopores. Detectors operably coupled to the nanopores may detect the labeled nucleotides. By determining the time intervals at which labeled nucleotides are detected, distance maps for each type of labeled nucleotide may be compiled. The distance maps in turn may be used to sequence and/or identify the nucleic acid. In different embodiments of the invention, luminescent nucleotides or nanoparticles may be detected using photodetectors or electrical detectors. Apparatus and sub-devices of use for nucleic acid sequencing and/or identification are also disclosed herein.

### RELATED APPLICATIONS

The present application is a continuation-in-part of pending U.S. patent application Ser. No. 10/153,125, filed on May 20, 2002.

### FIELD

The claimed apparatus and methods relate to the analysis of analytes including, but not limited to, nucleic acids. In particular, the apparatus and methods relate to nucleic acid sequencing and/or identification.

### BACKGROUND

Genetic information is stored in the form of very long molecules of deoxyribonucleic acid (DNA), organized into chromosomes. The human genome contains approximately three billion bases of DNA sequence. This DNA sequence information determines multiple characteristics of each individual. Many common diseases are based at least in part on variations in DNA sequence.

Determination of the entire sequence of the human genome has provided a foundation for identifying the genetic basis of such diseases. However, a great deal of work remains to be done to identify the genetic variations associated with each disease. That would require DNA sequencing of portions of chromosomes in individuals or families exhibiting each such disease, in order to identify specific changes in DNA sequence that promote the disease. Ribonucleic acid (RNA), an intermediary molecule in processing genetic information, may also be sequenced to identify the genetic bases of various diseases.

Existing methods for nucleic acid sequencing, based on detection of fluorescently labeled nucleic acids that have been separated by size, are limited by the length of the nucleic acid that can be sequenced. Typically, only 500 to 1,000 bases of nucleic acid sequence can be determined at one time. This is much shorter than the length of the functional unit of DNA, referred to as a gene, which can be tens or even hundreds of thousands of bases in length. Using current methods, determination of a complete gene sequence requires that many copies of the gene be produced, cut into overlapping fragments and sequenced, after which the overlapping DNA sequences may be assembled into the complete gene. This process is laborious, expensive, inefficient and time-consuming. It also typically requires the use of fluorescent or radioactive labels, which can potentially pose safety and waste disposal problems.

More recently, methods for nucleic acid sequencing have been developed involving hybridization to short oligonucleotides of defined sequenced, attached to specific locations on DNA chips. Such methods may be used to infer short nucleic acid sequences or to detect the presence of a specific nucleic acid in a sample, but are cumbersome and time-consuming for sequencing long nucleic acids, requiring fragmentation of multiple copies of the target nucleic acid into overlapping pieces a few hundred bases long.

### BRIEF DESCRIPTION OF THE DRAWINGS

The following drawings form part of the specification and are included to further demonstrate certain aspects of the disclosed methods and apparatus. The methods and apparatus may be better understood by reference to one or more of these drawings in combination with the detailed description presented herein.

FIG. 1 is a flow chart illustrating an exemplary apparatus 100 (not to scale) and methods for nucleic acid sequencing 150 and/or identification 160 by generation of distance maps 140.

FIG. 2 illustrates a non-limiting example of a sub-device 200 (not to scale) for nucleic acid sequencing and/or identification by photodetection.

FIG. 3 illustrates another non-limiting example of a sub-device 300 (not to scale) for nucleic acid sequencing and/or identification by electrical detection.

FIG. 4 shows exemplary methods for nucleic acid tagging.

FIG. 5 shows the Raman spectra of all four deoxynucleoside monophosphates (dNMPs) at 100 mM concentration. Characteristic Raman emission peaks were observed for each different type of nucleotide. The data were collected without surface-enhancement or labeling of the nucleotides.

FIG. 6 shows a comparative SERS spectrum of a 500 nM solution of deoxyadenosine triphosphate covalently labeled with fluorescein (upper trace) and unlabeled dATP (lower trace). The dATP-fluorescein was obtained from Roche Applied Science (Indianapolis, Ind.). A strong increase in the SERS signal was detected in the fluorescein labeled dATP.

FIG. 7 shows the SERS detection of a 0.9 nM (nanomolar) solution of adenine. The detection volume was 100 to 150 femtoliters, containing an estimated 60 molecules of adenine.

FIG. 8 shows the SERS detection of a rolling circle amplification product, using a single-stranded, circular M13 DNA template.

FIG. 9 illustrates exemplary methods for tagging nucleic acids on thiol moieties.

FIG. 10 illustrates exemplary methods for tagging nucleic acids on amine moieties.

FIG. 11 illustrates exemplary methods for tagging nucleic acids on carboxyl moieties.

FIG. 12 shows the Raman spectra of exemplary labeled oligonucleotides.

FIG. 13 illustrates an exemplary apparatus for nucleic acid analysis.

FIG. 14 shows another exemplary method for nucleic acid tagging.

### DETAILED DESCRIPTION

Definitions

As used herein, “a” or “an” may mean one or more than one of an item.

The terms “nanopore”, “nanochannel,” and “nanotube” refer respectively to a hole, channel or tube with a diameter or width of between 1 and 999 nanometers (nm). In a non-limiting example, the diameter is between 1 and 100 nm. As used herein, the terms “nanopore”, “nanotube” and “nanochannel” may be used interchangeably. The skilled artisan will realize that where the specification refers to a “nanopore,” different alternatives may use a “nanochannel” or “nanotube.” The only requirement is that the nanopore, nanochannel or nanotube connect one fluid filled compartment to another and allow the passage and detection of labeled nucleic acids.

As used herein, “operably coupled” means that there is a functional and/or structural relationship between two or more units. For example, a detector may be “operably coupled” to a nanopore if the detector is arranged so that it may identify labeled nucleotides passing through the nanopore. Similarly, a nanopore may be operably coupled to a chamber if nucleic acids in the chamber can pass through the nanopore. A detector may also be “operably coupled” to a nanopore where the detector and/or sensing elements of the detector are integrated into the nanopore.

As used herein, “fluid communication” refers to a functional connection between two or more compartments that allows fluids to pass between the compartments. For example, a first compartment is in “fluid communication” with a second compartment if fluid may pass from the first compartment to the second and/or from the second compartment to the first compartment.

“Nucleic acid” encompasses DNA, RNA, single-stranded, double-stranded or triple-stranded and any chemical modifications thereof. Virtually any modification of the nucleic acid is contemplated. A “nucleic acid” may be of almost any length, from 10, 20, 50, 100, 200, 300, 500, 750, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 2,000,000, 5,000,000 or even more bases in length, up to a full-length chromosomal DNA molecule.

A nucleoside is a molecule comprising a purine or pyrimidine base, such as adenine—“A”, thymine—“T”, guanine—“G”, cytosine—“C” or uracil—“U”, covalently attached to a pentose sugar, such as deoxyribose, ribose or derivatives or analogs of pentose sugars. A “nucleotide” refers to a nucleoside further comprising at least one phosphate group covalently attached to the pentose sugar. It is contemplated that various substitutions or modifications may be made in the structure of the nucleotides, so long as they are still capable of being incorporated into a complementary nucleic acid by a polymerase. For example, the ribose or deoxyribose moiety may be substituted with another pentose sugar or a pentose sugar analog. The phosphate groups may be substituted by various groups, such as phosphonates, sulphates or sulfonates. The purine or pyrimidine bases may be substituted by other purines or pyrimidines or analogs thereof, so long as the sequence of nucleotides incorporated into a complementary nucleic acid strand reflects the sequence of the template strand.

### DESCRIPTION OF ILLUSTRATIVE EMBODIMENTS

The disclosed methods and apparatus are of use for the rapid, automated sequencing and/or identification of nucleic acid molecules. Advantages over prior art methods include: high throughput, as fast as 3×10⁶ bases per second (>3×10⁷ times faster than current methods); ultra-sensitive detection of single labeled nucleic acid molecules; nanometer scale resolution of nucleic acid base distances; and lower unit cost of nucleic acid sequencing and/or identification.

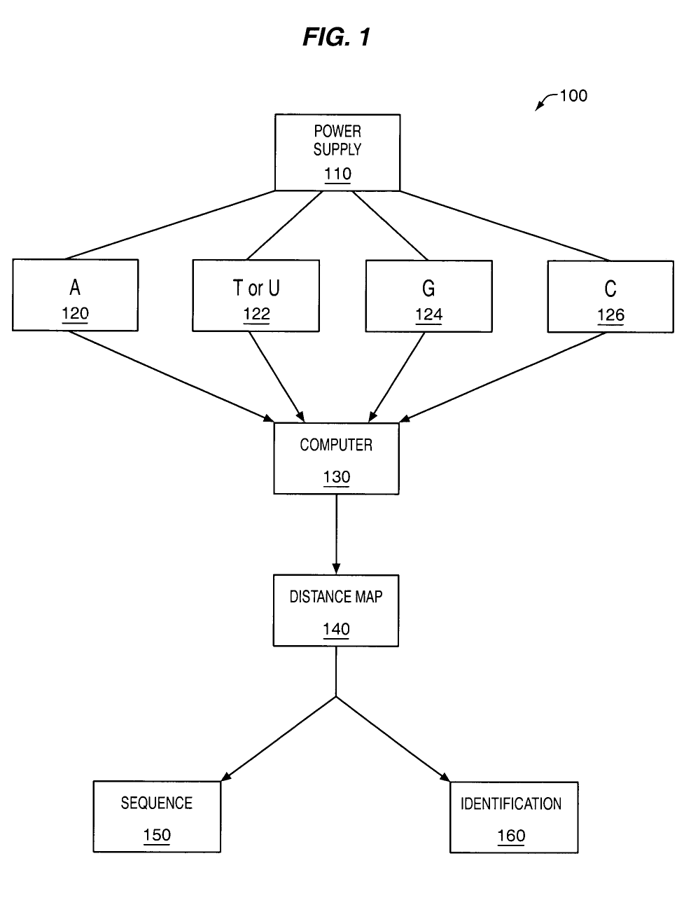

As illustrated in FIG. 1, a template nucleic acid may be placed into four chambers 120, 122, 124, 126, each chamber 120, 122, 124, 126 to contain a different labeled nucleotide—A, G, C and T or U. Labeled complementary nucleic acid strands may be synthesized from the template nucleic acids using known synthetic techniques, as discussed below. The labeled nucleic acids from each chamber 120, 122, 124, 126 may pass through one or more nanopores operably coupled to detectors that can detect labeled nucleotides. Each chamber 120, 122, 124, 126 is associated with a different set of nanopores. The distances between labeled nucleotides are measured to compile a map of distances 140 for each type of labeled nucleotide. The distance maps 140 are used to identify 160 and/or sequence 150 the template nucleic acid.

The skilled artisan will realize that the distance maps 140 of use may show distances in the sub-nanometer or greater scale. For example, a single nucleotide in a linear nucleic acid sequence would have a size of about 0.9 nm. During typical gel electrophoresis of nucleic acids (field strength of about 10 volt/cm), molecules may travel about 100 mm in 60 minutes (or about 28,000 nm per second). Since currently available electrical detectors are capable of counting down to the femto second scale, detection of adjacent nucleotides is well within the detection limits. Given the mobility rate of nucleic acids under electrophoresis, a 1 nanosecond time frame would be equivalent to a distance of 0.036 nm, which is less than the carbon-carbon bond length of about 0.154 nm. It would take about 30 nanoseconds to detect two adjacent nucleotide residues. The distance maps 140 may range from the average subunit distance (0.6 nm) up to the length of a full-length nucleic acid.

The nanopore may be of a diameter that restricts passage to an individual single- or double-stranded nucleic acid molecule. In such case, only one labeled nucleic acid may pass through a nanopore at one time. The skilled artisan will realize that although various parts of the instant disclosure refer to nanopores, the disclosed methods and apparatus could utilize nanochannels or nanotubes in place of the nanopores.

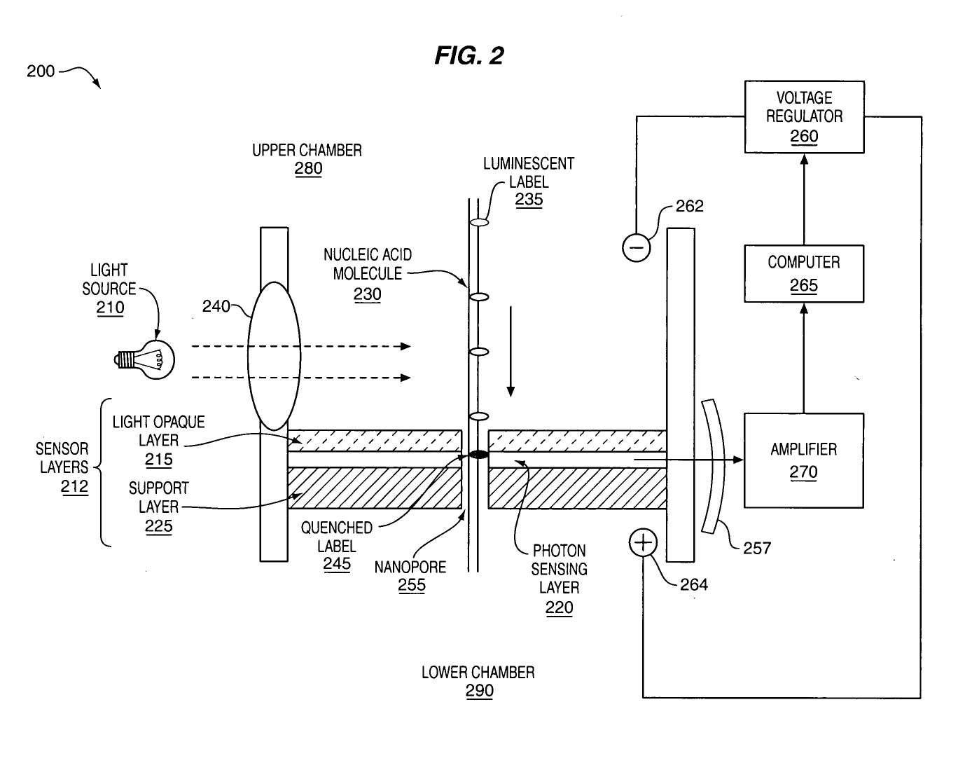

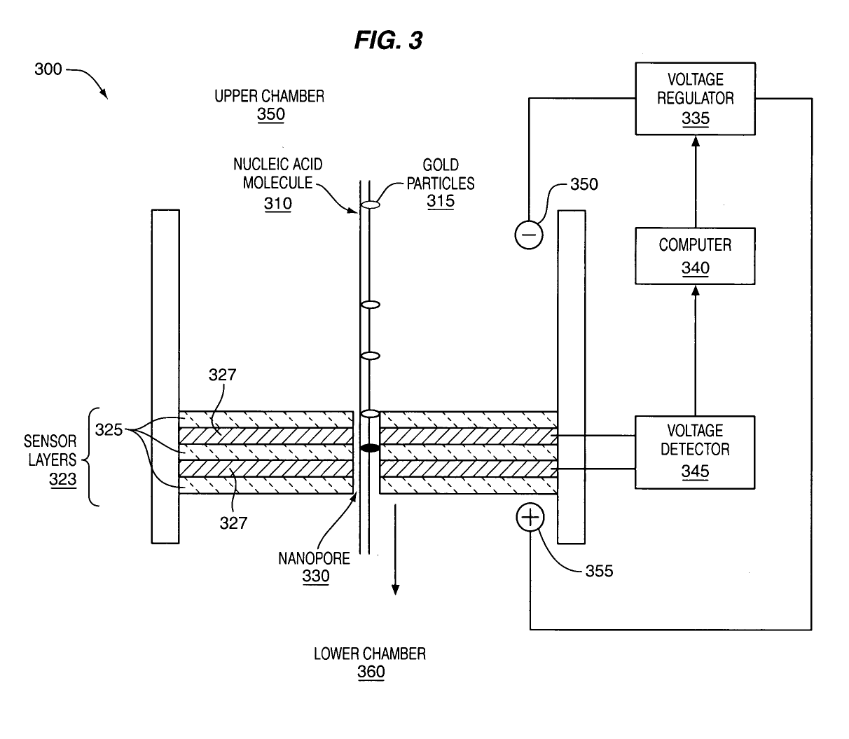

As illustrated in FIG. 2 and FIG. 3, an apparatus may comprise one or more sub-devices 200, 300. Each sub-device 200, 300 may comprise fluid filled first 280, 350 and second 290, 360 chambers, separated by sensor layers 212, 323. One or more nanopores 255, 330 may extend through the sensor layers 212, 323 and allow passage of nucleic acids 230, 310. The nanopores 255, 330 may be operably coupled to one or more detectors 257, 345 that can detect labeled nucleotides 235, 245, 315 as they pass through the nanopores 255, 330. Electrodes 262, 264, 350, 355 in the first and second chambers 280, 350, 290, 360 may generate an electrical field that drives labeled nucleic acids 230, 310 from the first 280, 350 to the second chamber 290, 360 through the nanopores 255, 330. The electrical gradient may be controlled by a voltage regulator 260, 335, which may be operably coupled to a computer 265, 340. The nature of the electrical gradient is not limiting and the applied voltage may be alternating current, direct current, pulse field direct current, reverse phase current or any other known type of electrical gradient.

Detection may occur by photodetection or electrical detection. Where photodetection is used (FIG. 2), the sensor layers 212 may comprise one or more support layers 225, photon-sensing layers 220, and light opaque layers 215. Nucleotides labeled with a photolabel 235 may be excited by a light source 210, such as a laser. Excitatory light may pass through a transparent window 240 in the first chamber 280, exciting the photolabel 235 to a higher energy state. The window 240 may comprise one or more filters and/or lenses to focus the excitatory light. The labeled nucleotides 235, 245 may pass through the light opaque layer 215, cutting off the source 210 of excitatory light and shielding the photodetector 257 from the light source 210. As the photolabel 235 passes the photon sensing layer 220, it emits a photon and returns to an unexcited state 245. In alternative embodiments involving fluorescence resonance energy transfer FRET, the energy of the excited photolabel 235 (donor molecule) may be non-radiatively transferred to one or more fixed fluorescence acceptor molecules located at the photon sensing layer 220.

The excited acceptor molecule may emit a photon. The emitted photon may be transmitted through the photon sensing layer 220 to a photodetector 257, where the signal is detected. The detected signal may be amplified by an amplifier 270 and stored and/or processed by a computer 265. The computer 265 may also record the time at which each labeled nucleotide 235, 245 passes through the nanopore 255, allowing the calculation of distances between adjacent labeled nucleotides 235, 245 and the compilation of distance maps for the distances between different types of labeled nucleotides 235, 245. Photon sensing layers may be comprised of any material that is relatively translucent at the wavelengths of light emitted by the photolabel 235, 245 for example glass, silicon or certain types of plastics.

A wide variety of materials and structures are of use for photon sensing layers 220. In certain non-limiting examples, the photon sensing layer 220 may serve to simply conduct light to the photon sensing elements of a photodetector 257. In other alternatives, the photon sensing element may be integrated into the nanopore 255. For example, a photon sensitive PN junction may be directly fabricated into the photon sensing layer 220 surrounding a nanopore 255 by layering with different types of materials (e.g., P-doped and N-doped silicon or gallium arsenide (GaAs)) or by coating the inner surface of the nanopore 255 with a different type of semiconductor material. Methods for forming layers of P-doped and N-doped semiconductors are well known in the arts of computer chip and/or optical transducer manufacture. The effects of various dopants, such as P, As, Sb, Se, Ge, Sn, Be, B, Mg, Zn and C on semiconductor properties are also known in the art. A photon transducer transduces a photonic signal into an electrical signal counterpart. Different types of known photon transducing structures that may be used to detect light emission include those based on photoconductive materials, photovoltaic cells (photocells), photoemissive materials (photomultiplier tubes, phototubes) and semiconductor pn junctions (photodiodes).

In a photoconductive cell, a semiconductor such as CdS, PbS, PbSe, InSb, InAs, HgCdTe or PbSnTe, behaves like a resistor. The semiconductor is in series with a constant voltage source and a load resistor. The voltage across the load resistor is used to measure the resistance of the semiconductor material. Incident radiation, for example in the form of an emitted photon from a tagged nucleotide residue, causes band-gap excitation and lowers the resistance of the semiconductor.

A photodiode contains a reverse-bias semiconductor pn junction. The p-type semiconductor (e.g., boron doped silicon, beryllium doped GaAs) has excess electron holes, while the n-type semiconductor (e.g., phosphorus doped silicon, silicon doped GaAs) has excess electrons. Under a reverse bias, a depletion layer forms at the pn junction between the p-type and n-type semiconductors. A reverse bias is initiated when an external electrical potential is applied that forces electron holes in the p-type semiconductor and excess electrons in the n-type semiconductor to migrate away from the pn junction. When the material is irradiated, electron-hole pairs are formed that move under bias, resulting in a temporary electrical current across the pn junction. Photodiodes and other types of photon transducing structures may be incorporated into a nanopore 255 and used as photon sensing elements of a photodetector 257.

Where electrical detection is used (FIG. 3), the sensor layers 325 may comprise at least two insulating layers 325 and at least one conducting layer 327. Typically, insulating layers 325 would be exposed to the medium in the first 350 and second 360 buffer chambers, insulating the conducting layers 327 from the external electrical field imposed by the electrodes 350, 355. The conducting layer 327 may be operably coupled to an electrical detector 345, which may detect any type of electrical signal, such as voltage, conductivity, resistance, impedance, capacitance, etc. The nucleotides may be tagged with a label 315 that can be detected by its electrical properties. In one non-limiting example, the label 315 may comprise gold nanoparticles. As a nucleotide labeled with a gold nanoparticle 315 passes through the nanopore 330, it produces changes in the conductivity, resistance and other electrical properties of the nanopore 330 compared to unlabeled portions of the nucleic acid 310. Thus, passage of labeled nucleotides 315 through the nanopore 330 may be detected by the electrical detector 345. Signals detected by the electrical detector 345 may be processed and/or stored by a computer 340. Distance maps between labeled nucleotides 315 may be compiled and the nucleic acid 310 sequenced and/or identified.

Nanopores, Nanochannels and Nanotubes

Size Characteristics

In certain non-limiting examples, the nanopores may be 0.5, 1, 2, 3, 4, 5, 6, 7, 8, 9 or 10 nm in diameter. Alternatively, the diameter may range between 1-3, 1-5, 1-10, 1-20, 1-50, 1-100, 5-10, 5-20, 10-20, 20-50, 30-75, 50-75, 50-100, 75-100, 200-300, 300-400, 400-500 or 100-999 nm. A nanopore of approximately 2.6 nm will permit passage of an individual nucleic acid molecule. Where the nucleotides are labeled with bulky groups, the nanopores may be larger to allow passage of labeled nucleic acids. In alternatives that utilize nanotubes or nanochannels in place of nanopores, the same size ranges apply to the diameter or width of the nanotubes or nanochannels.

Fabrication

Fabrication of nanopores, nanotubes and/or nanochannels, individually or in arrays, may utilize any technique known in the art for nanoscale manufacturing. The following techniques are exemplary only. Nanopores, nanochannels and/or nanotubes may be constructed on a solid-state matrix comprising sensor layers by known nanolithography methods, including but not limited to chemical vapor deposition, electrochemical deposition, chemical deposition, electroplating, thermal diffusion and evaporation, physical vapor deposition, sol-gel deposition, focused electron beam, focused ion beam, molecular beam epitaxy, dip-pen nanolithography, reactive-ion beam etching, chemically assisted ion beam etching, microwave assisted plasma etching, electro-oxidation, scanning probe methods, chemical etching, laser ablation, or any other method known in the art (E.g., U.S. Pat. No. 6,146,227).

The sensor layers may comprise semiconductor materials including, but not limited to, silicon, silicon oxide, silicon dioxide, germanium, gallinium arsenide, and metal-based compositions such as metals and/or metal oxides. Sensor layers may be processed by electronic beam, ion beam and/or laser lithography and etching to create a channel, groove, or hole. The channel, hole or groove may be coated with an organic or inorganic deposit to reduce the diameter of the channel, hole or groove, or to endow the resultant nanopore, nanotube and/or nanochannel with certain physico-chemical characteristics, such as hydrophilicity. Conducting layers comprising metals may be deposited onto a semiconductor surface by means of field evaporation from a scanning tunnel microscopy (STM) or atomic force microscopy (AFM) tip or from a solution or vapor or other known methods of metal deposition. Insulating layers may be formed by oxidizing the semiconductor's surface to an insulating composition or by deposition of known insulators.

Channels or grooves may be etched into a semiconductor surface by various techniques known in the art including, but not limited to, methodologies using an STM/AFM tip in an oxide etching solution. After channels are formed, two semiconductor surfaces may be opposed to create a plurality of nanopores that penetrate the semiconductor. Such nanopores may be of a size that restricts passage to single nucleic acid molecules. STM tip methodologies may be used to create nanopores, nanodetectors, nanosensors, nanowires, nanoleads, nanochannels, and other nanostructures using techniques known in the art. Alternatively, scanning probes, chemical etching techniques, and/or micromachining may be used to cut micrometer-dimensioned or nanometer-dimensioned channels, grooves or holes in a semiconductor substrate.

Nano-molding may also be employed, wherein formed nanotubes, such as carbon or metallic nanotubes, are placed or grown on a semiconductor chip substrate. After depositing layers on the substrate, the nanotubes may be removed, leaving a nanochannel and/or nanopore imprint in the substrate material. Such nanostructures can be built in clusters with properties of molecular electrodes that may function as detectors on a chip.

Nanopores and/or nanochannels may also be made using a high-throughput electron-beam lithography system. Electron-beam lithography may be used to write features as small as 5 nm on silicon chips. Sensitive resists, such as polymethyl-methacrylate, coated on silicon surfaces may be patterned without use of a mask. The electron-beam array may combine a field emitter cluster with a microchannel amplifier to increase the stability of the electron beam, allowing operation at low currents. The SoftMask™ computer control system may be used to control electron-beam lithography of nanoscale features on a semiconductor chip substrate.

Alternatively, nanopores and/or nanochannels may be produced using focused atom lasers (e.g., Bloch et al., “Optics with an atom laser beam,” Phys. Rev. Lett. 87:123-321, 2001). Focused atom lasers may be used for lithography, much like standard lasers or focused electron beams. Such techniques are capable of producing micron scale or even nanoscale structures on a chip. In other alternatives, dip-pen nanolithography may be used to form nanopores and/or nanochannels (e.g., Ivanisevic et al., “Dip-Pen Nanolithography on Semiconductor Surfaces,” J. Am. Chem. Soc., 123: 7887-7889, 2001). Dip-pen nanolithograpy uses AFM techniques to deposit molecules on surfaces, such as silicon chips. Features as small as 15 nm in size may be formed, with spatial resolution of 10 nm. Nanoscale pores and/or channels may be formed by using dip-pen nanolithography in combination with regular photolithography techniques. For example, a micron scale line in a layer of resist may be formed by standard photolithography. Using dip-pen nanolithography, the width of the line and the corresponding diameter of the channel after etching may be narrowed by depositing additional resist compound. After etching of the thinner line, a nanoscale channel may be formed. Alternatively, AFM methods may be used to remove photoresist material to form nanometer scale features.

In other alternatives, ion-beam lithography may be used to create nanopores and/or nanochannels on a chip (e.g., Siegel, “Ion Beam Lithography,” VLSI Electronics, Microstructure Science, Vol. 16, Einspruch and Watts eds., Academic Press, New York, 1987). A finely focused ion beam may be used to write nanoscale features directly on a layer of resist without use of a mask. Alternatively, broad ion beams may be used in combination with masks to form features as small as 100 nm in scale. Chemical etching, for example, with hydrofluoric acid, may be used to remove exposed silicon or other chip material that is not protected by resist. The skilled artisan will realize that the techniques disclosed above are not limiting, and that nanopores and/or nanochannels may be formed by any method known in the art.

Carbon Nanotubes

Nanopores may comprise, be attached to or be replaced by nanotubes, such as carbon nanotubes. The carbon nanotubes may be coated with an organic or inorganic composition, leaving a deposited layer “mold” on the carbon nanotube. When the nanotube is removed and separated from the organic or inorganic deposit, a nanopore may be created in the “mold.” Carbon nanotubes may be formed in a semiconductor with other components, such as sensor layers formed around the nanotubes.

Carbon nanotubes may be manufactured by chemical vapor deposition (CVD), using ethylene and iron catalysts deposited on silicon (e.g., Cheung et al. PNAS 97: 3809-3813, 2000). Single-wall carbon nanotubes may be formed on silicon chips by CVD using AFM Si₃N₄ tips (e.g., Cheung, et al., 2000; Wong, et al. Nature 394: 52-55, 1998). A flat surface of 1-5 μm² may be created on the silicon AFM tips by contact with silicon or CVD diamond surfaces (GE Suprabrasives, Worthington, Ohio) at high load (˜1 μN), at high scan speed (30 Hz), and with a large scan size (40 μm) for several minutes. Approximate 100 nm diameter, 1 μm deep pores in the ends of the AFM tips may be made by anodization at 2.1 V for 100 sec. Anodized tips may be etched in 0.03% KOH in water for 50 sec, after which excess silicon may be removed and nanopores opened at the surface of the tip.

Carbon nanotubes may be attached to AFM tips using known methods. For example, iron catalyst consisting of iron oxide nanoparticles may be synthesized according to Murphy et al. (Austr. J. Soil Res. 13:189-201, 1975). Iron catalyst (0.5 to 4 nm particles) may be electrochemically deposited from a colloidal suspension into the pores using platinum counter electrodes at −0.5 V (Cheung, et al., 2000). Tips may be washed in water to remove excess iron oxide particles. AFM tips may be oxidized by heating in oxygen gas and carbon nanotubes may be grown on the catalyst by controlled heating and cooling in the presence of a carbon source (Murphy et al., 1975; Cheung et al., 2000). The diameter of the resulting nanotubes should correspond to the size of the iron oxide catalyst used (0.5 to 4 nm). Individual, single-walled nanotubes prepared under these conditions are aligned perpendicular to the flattened surface of the AFM tip.

Nanotubes may be cut to a predetermined length using known techniques. For example, carbon nanotubes may be attached to pyramids of gold-coated silicon cantilevers using an acrylic adhesive. The carbon nanotubes may be shortened to a defined length by application of a bias voltage between the tip and a niobium surface in an oxygen atmosphere (Wong, et al., Nature 394:52-55, 1998). Alternatively, high-energy beams may be used to shorten carbon nanotubes. Such high energy beams may include, but are not limited to, laser beams, ion beams, and electron beams. Other methods for truncating carbon nanotubes are known (e.g., U.S. Pat. No. 6,283,812). Preformed carbon nanotubes may be attached to a chip material such as silicon, glass, ceramic, germanium, polystyrene, and/or gallium arsenide (e.g., U.S. Pat. Nos. 6,038,060 and 6,062,931).

A first set of carbon nanotubes may be used as cold cathode emitters on semiconductor chips, associated with a second set of nanotubes containing nucleic acids. The first set of nanotubes may be used to create local electrical fields of at least 10⁶ volts/cm, when an external voltage of between 10 and 50 volts is applied. Such an electric field in the first set of nanotubes may be used to drive nucleic acids through the second set of nanotubes, or to generate an electrical or electromagnetic signal to detect labeled nucleotides (see Chuang, et al., 2000; U.S. Pat. No. 6,062,931). A first set of nanotubes that act as detectors, electromagnetic conduits or optical devices may be operably coupled to a second set of nanotubes containing labeled nucleic acids. The nanotubes may be placed in operable contact with each other or with other elements such as detectors by known nanomanipulation techniques. Each nanotube in the first set may be operably coupled to a nanotube in the second set, such that the nanotubes are positioned perpendicular to or otherwise arranged with respect to each other.

Electromagnetic radiation from a third set of nanotubes may excite a light-sensitive (e.g., luminescent, fluorescent, phosphorescent) label attached to a nucleic acid passing through a second set of nanotubes, leading to emission of light detected by a photodetector that is operably coupled to a first set of nanotubes.

Ion Channels on Semiconductor Chips

Nanopores may be single ion channels in lipid bilayer membranes (e.g., Kasianowitz, et al., Proc. Natl. Acad. Sci. USA 93:13770-13773, 1996). Such ion channels may include, but are not limited to, Staphylococcus aureus alpha-hemolysin and/or mitochondrial voltage-dependent anion channels. These ion channels may remain open for extended periods of time, allowing continuous current to flow across the lipid bilayer. An electric field applied to single-stranded RNA and DNA molecules may cause these molecules to move through ion channels in lipid bilayer membranes (Kasianowitz et al., 1996). Single-stranded nucleic acids may pass through the ion channel in linear fashion. Ion channels may be incorporated into chips and operably coupled to detectors.

Micro-Electro-Mechanical Systems (MEMS)

Micro-Electro-Mechanical Systems (MEMS) are integrated systems comprising mechanical elements, detectors, switches, diodes, transistors, valves, gears, mirrors, actuators, and electronics. All of those components may be manufactured by known microfabrication techniques on a common chip, comprising a silicon-based or equivalent substrate (e.g., Voldman et al., Ann. Rev. Biomed. Eng. 1:401-425, 1999). The detector component of MEMS may be used to measure mechanical, thermal, biological, chemical, optical and/or magnetic phenomena. The electronics may process the information from the sensors and control actuator components such pumps, valves, heaters, coolers, filters, etc. thereby controlling the function of the MEMS.

The electronic components of MEMS may be fabricated using integrated circuit (IC) processes (e.g., CMOS, Bipolar, or BICMOS processes). They may be patterned using photolithographic and etching methods known for computer chip manufacture. The micromechanical components may be fabricated using compatible “micromachining” processes that selectively etch away parts of the silicon wafer or add new structural layers to form the mechanical and/or electromechanical components. Basic techniques in MEMS manufacture include depositing thin films of material on a substrate, applying a patterned mask on top of the films by photolithographic imaging or other known lithographic methods, and selectively etching the films. A thin film may have a thickness in the range of a few nanometers to 100 micrometers. Deposition techniques of use may include chemical procedures such as chemical vapor deposition (CVD), electrodeposition, epitaxy and thermal oxidation and physical procedures like physical vapor deposition (PVD) and casting. Sensor layers of 5 nm thickness or less may be formed by such known techniques. Standard lithography techniques may be used to create sensor layer areas of micron or sub-micron dimensions, operably connected to detectors and nanopores.

The manufacturing method is not limiting and any methods known in the art may be used, such as atomic layer deposition, pulsed DC magnetron sputtering, vacuum evaporation, laser ablation, injection molding, molecular beam epitaxy, dip-pen nanolithograpy, reactive-ion beam etching, chemically assisted ion beam etching, microwave assisted plasma etching, focused ion beam milling, electron beam or focused ion beam technology or imprinting techniques. Methods for manufacture of nanoelectromechanical systems may be used for certain types of structures. (See, e.g., Craighead, Science 290:1532-36, 2000.) Various forms of microfabricated chips are commercially available from, e.g., Caliper Technologies Inc. (Mountain View, Calif.) and ACLARA BioSciences Inc. (Mountain View, Calif.).

It is contemplated that some or all of the components of a nucleic acid sequencing apparatus may be constructed as part of an integrated MEMS device. Nanoelectrodes comprising conducting metals such as gold, platinum, or copper may be operably coupled to nanopores, nanochannels and/or nanotubes using STM technologies known in the art (e.g., Kolb et al., Science 275:1097-1099, 1997). Nanoelectrodes, detectors and other components may be connected by nanowires.

Standard photolithography may be used to create an array of multiplaner structures (0.5×0.5 μm) on a silica substrate, each structure with a silica base support and two layers of gold films separated by an insulator layer comprising silica oxide, with another insulator layer overlaying the top gold film. A chip containing the structures may be divided in half and placed on its side. A thin layer of resist may be coated on the sides of the chip, perpendicular to the conducting and insulating layers. An AFM/STP tip may be used to etch 5-10 nm lines in the resist layer, overlaying each structure. Chemical etching may be used to create nano-scale grooves in each of the structures. When the halves of the chip are aligned and fused together, the grooves may form nanopores and/or nanochannels, which extend through the sensor layers. Nanowires connecting the conducting layers to electrical detectors may be formed by known methods discussed above. The nanowires may be used to apply a voltage across the conducting layers and changes in current, resistance or other electrical properties may be detected with the passage of a nucleic acid through the nanopore. A thin layer of insulating material may be coated onto the sides of the divided chip, forming a barrier that prevents current flow except through the nanopore.

Where photodetection is used instead of electrical detection, the conducting and insulating layers may be replaced with light opaque and photon sensing layers. Polymeric materials may be coated onto the chip to enhance detectability of signals. Such polymeric materials may include, but are not limited to, polymethylmethacrylate, ultraviolet-curable polyurethanes and epoxies, and other polymers that exhibit optical transparency, low fluorescence at excitation wavelengths, electrical conductivity and/or insulation. Such materials may be formed into appropriate structures, for example by polymer casting and chemical or photochemical curing (Kim et al., Nature 376: 581-584 1995).

Detectors

Electrical Detectors

An electrical detector may detect electrical signals induced in a conducting layer as a function of the passage of a labeled nucleic acid through a nanopore. Non-limiting examples of electrical signals include induced current, voltage, impedance, induced electromotive force, signal sign, frequency or noise signature of a predetermined electrical signal generated at one location and received at another location. An electrical detector may be operably coupled to one or more conducting layers, a power supply and one or more nanopores perpendicular to and penetrating the conducting layers. The detector may comprise an ammeter, voltmeter, capacitance meter and/or conductivity meter to measure induced current, voltage, resistance, etc. Other electrical components such as resistors and/or capacitors may be included in an electrical circuit associated with the detector.

The first and second chambers may be filled with a low conductivity aqueous buffer. An electrical potential may be applied to the conducting layers flanking a nanopore. When buffer alone is present, the resistance between the conducting layers is high. The presence of unlabeled regions of nucleic acids passing through the nanopore would produce a slight increase in conductivity across the nanopore, due to the present of conjugated pi electrons and charged groups, such as phosphates. The passage of nucleotides labeled with highly conductive labels, such as metal nanoparticles, would result in a large increase in conductivity that produces a detectable signal at the detector. In a non-limiting example, the nanoparticle labels may be about 1 nm diameter gold nanoparticles, although other sizes and compositions of nanoparticles may be used. The time interval between electrical signals may be measured and used to create a distance map representing the positions of labeled nucleotides on the nucleic acid molecule. By compiling such maps for each of the four types of labeled nucleotides in the different sub-chambers, a complete sequence of the nucleic acid may be generated, or the nucleic acid may be identified.

The skilled artisan will realize that other labeling schemes may be used within the scope of the disclosed methods. For example, nucleic acids may be identified by labeling a single type of nucleotide, such as adenosine (A) residues. Over a long enough sequence, target nucleic acids would be expected to exhibit unique patterns (distances) of labeled adenosine residues. Similarly, the skilled artisan will realize that sequence data may be generated by labeling less than all four types of nucleotides. For example, the two strands of double-stranded DNA may be labeled only on C and T residues, or only on A and G residues. Because the strands are complementary, with C paired with G and T paired with A, it is possible to label both strands on only purine or pyrimidine residues, obtain distance maps for the two strands, and generate a complete nucleic acid sequence by combining the distance maps for the two complementary strands.

The first and second chambers may be filled with a solution of 0 to 10 mM KCl, 5 mM Hepes pH 7.5. A 2 to 3 nm nanopore may provide fluid communication between the first and second chambers. Nucleic acids labeled with 1 nm gold nanoparticles may be synthesized and/or placed in the first chamber. A detector and power supply may be operably coupled to conducting layers flanking the nanopore. Current across the nanopore may be converted to voltage and amplified using an Axopatch 200A (Axon Instruments, Foster City, Calif.) or a Dagan 3900A patch clamp amplifier (Dagan Instruments, Minneapolis, Minn.). The signal may be filtered using a Frequency Devices (Haverhill, Mass.) low pass Bessel filter. Data may be digitized using a National Instruments (Austin, Tex.) AT-MIO-16-X 16-bit board and LAB WINDOWS/CVI programs. The chip may be shielded from electric and magnetic noise sources using a mu-metal box (Amuneal, Philadelphia, Pa.) (see Kasianowicz, et al., 1996).

In this non-limiting example, the absence of a nucleic acid in the nanopore may result in single channel currents that are free of transient fluctuations when a potential of about −120 mV is applied. After entry of the nucleic acid molecule into the nanopore, current blockage patterns may be measured. Labeled nucleotides attached to 1.0 nm gold particles may exhibit greater current fluctuations that are detectable over unlabeled nucleic acid regions. Nucleic acid sequences may be obtained by comparing distance maps for each sub-chamber.

Spectrophotometric Detection

Alternatively, labeled nucleotides may be detected using a light source and photodetector, such as a diode-laser illuminator and fiber-optic or phototransistor detector. (E.g., Sepaniak et al., J. Microcol. Separations 1:155-157, 1981; Foret et al., Electrophoresis 7:430-432, 1986; Horokawa et al., J. Chromatog. 463:39-49 1989; U.S. Pat. No. 5,302,272.) Other exemplary light sources of use include vertical cavity surface-emitting lasers, edge-emitting lasers, surface emitting lasers and quantum cavity lasers, for example a Continuum Corporation Nd-YAG pumped Ti:Sapphire tunable solid-state laser and a Lambda Physik excimer pumped dye laser. Other exemplary photodetectors include photodiodes, avalanche photodiodes, photomultiplier tubes, multianode photomultiplier tubes, phototransistors, vacuum photodiodes, silicon photodiodes, and charge-coupled devices (CCDs).

A photodetector, light source, and nanopore may be fabricated into a semiconductor chip using known N-well Complementary Metal Oxide Semiconductor (CMOS) processes (Orbit Semiconductor, Sunnyvale, Calif.). Alternatively, the detector, light source and nanopore may be fabricated in a silicon-on-insulator CMOS process (e.g., U.S. Pat. No. 6,117,643). In other alternatives, an array of diode-laser illuminators and CCD detectors may be placed on a semiconductor chip using known techniques (U.S. Pat. Nos. 4,874,492 and 5,061,067; Eggers et al., BioTechniques 17: 516-524, 1994).

A highly sensitive cooled CCD detector may be used as a photodetector. The cooled CCD detector has a probability of single-photon detection of up to 80%, a high spatial resolution pixel size (5 microns), and sensitivity in the visible through near infrared range. (Sheppard, Confocal Microscopy: Basic Principles and System Performance in: Multidimensional Microscopy, P. C. Cheng et al. eds., Springer-Verlag, New York, N.Y. pp. 1-51, 1994.) A coiled image-intensified coupling device (ICCD) may also be used as a photodetector that approaches single-photon counting levels (U.S. Pat. No. 6,147,198). A nanochannel plate may operate as a photomultiplier tube wherein a small number of photons trigger an avalanche of electrons that impinge on a phosphor screen, producing an illuminated image. This phosphor image may be sensed by a CCD chip region attached to an amplifier through a fiber optic coupler. A CCD detector on the chip may be sensitive to ultraviolet, visible, and/or infrared spectra light (U.S. Pat. No. 5,846,708).

A nanopore containing a labeled nucleic acid may be operably coupled to a light source and a photodetector on a semiconductor chip. The detector may be positioned perpendicular to the light source to minimize background light. The photons generated by excitation of the photolabel on the nucleic acid may be collected by a fiber optic. The collected photons may be transferred to a CCD detector on the chip and the light detected and quantified. The times at which labeled nucleotides are detected may be recorded and nucleotide distance maps may be constructed. Methods of placement of optical fibers on a semiconductor chip in operable contact with a CCD detector are known (U.S. Pat. No. 6,274, 320).

An avalanche photodiode (APD) may be used to detect low light levels. The APD process uses photodiode arrays for electron multiplication effects (U.S. Pat. No. 6,197,503). Light sources, such as light-emitting diodes (LEDs) and/or semiconductor lasers may be incorporated into semiconductor chips (U.S. Pat. No. 6,197,503). Diffractive optical elements that shape a laser or diode light beam may also be integrated into a chip.

A light source may produce electromagnetic radiation that excites a photosensitive label, such as fluorescein, attached to the nucleic acid. In a non-limiting example, an air-cooled argon laser at 488 nm may excite fluorescein-labeled nucleic acid molecules. Emitted light may be collected by a collection optics system comprising a fiber optic, a lens, an imaging spectrometer, and a 0° C. thermoelectrically cooled CCD camera. Alternative examples of fluorescence detectors are known in the art (e.g., U.S. Pat. No. 5,143,8545).

Raman Spectroscopy

In other alternatives, labeled nucleotides may be detected by Raman spectroscopy. Raman labels of use in spectrophotometric detection of labeled nucleic acids are well known in the art. (See, e.g., U.S. Pat. Nos. 5,306,403; 6,002,471; 6,174,677.) Labeled nucleotides may be excited with a laser, photodiode, or other light source and the excited nucleotide detected by a variety of Raman techniques, including but not limited to surface enhanced Raman spectroscopy (SERS), surface enhanced resonance Raman spectroscopy (SERRS) normal Raman scattering, resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy. In SERS and SERRS, the sensitivity of the Raman detection is enhanced by a factor of 10⁶ or more for molecules adsorbed on roughened metal surfaces, such as silver, gold, platinum, copper or aluminum surfaces. For such embodiments, portions of the nanopores and/or sensor layers may be coated with a Raman sensitive metal, such as silver or gold to provide an enhanced Raman signal. Alternatively, an enhanced Raman signal may be produced by nucleotides labeled with gold or silver nanoparticles.

FRET Detection

In still other alternatives, a nucleic acid may be identified or sequenced using fluorescence resonance energy transfer (FRET). FRET is a spectroscopic phenomenon used to detect proximity between fluorescent donor and acceptor molecules. The donor and acceptor pairs are chosen such that fluorescent emission from the donor overlaps the excitation spectrum of the acceptor. When the two molecules are associated at a distance of less than 100 Angstroms, the excited-state energy of the donor is transferred non-radiatively to the acceptor molecule. If the acceptor molecule is a fluorophore then its emission is enhanced. Compositions and methods of use of FRET with oligonucleotides are known (e.g., U.S. Pat. No. 5,866,336).

Donor fluorophore molecules may be attached to a nucleotide and the acceptor fluorophore molecules may be connected to a nanopore or sensor layers. Following excitation by a light source, the donor fluorophore molecules may transfer their energy to the acceptor molecules, resulting in an enhanced fluorescent signal from the acceptor molecules that may be detected by a photodetector.

Labels

Labeled nucleotides may be prepared by any method known in the art. A labeled nucleotide may be incorporated into a nucleic acid strand during synthesis, for example using a primer and DNA polymerase. Alternatively, labels may be attached by covalent, noncovalent, ionic, van der Waals, hydrogen bonding or other forces after nucleic acid synthesis. Nucleotide precursors incorporating reactive groups to which labels may be attached, such as sulfhydryl, carboxyl or amino residues, may be obtained from commercial sources. Following synthesis of a complementary nucleic acid strand incorporating the modified nucleotides, a variety of labels may be attached by standard chemistries. For example, gold nanoparticles may be covalently attached to sulfhydryl-modified nucleic acids by known techniques (e.g., Mirkin et al., Nature 382:581, 1996). Nucleic acids containing modified nucleotides with different functional groups, such as amine, carboxyl or sulfhydryl may be synthesized by alkynylamino-nucleotide chemistry as discussed below. Oligonucleotides synthesized with modified nucleotide residues are also commercially available (e.g., Midland Certified Reagents, Midland Tex.).

Detectable labels, may include, but are not limited to, any composition detectable by electrical, optical, spectrophotometric, photochemical, biochemical, immunochemical, or chemical techniques. Labels, may include, but are not limited to, conducting, luminescent, fluorescent, chemiluminescent, bioluminescent and phosphorescent labels, Raman labels, nuclear magnetic resonance labels, mass spectroscopy labels, electron spin resonance labels, electron paramagnetic resonance labels, nanoparticles, metal nanoparticles, gold nanoparticles, silver nanoparticles, chromogens, antibodies, antibody fragments, genetically engineered antibodies, enzymes, substrates, cofactors, inhibitors, binding proteins, magnetic particles and spin labels. (U.S. Pat. Nos. 3,817,837; 3,850,752; 3,939,350; 3,996,345; 4,277,437; 4,275,149; and 4,366,241.) Fluorescent molecules suitable for use as labels include fluorescein, dansyl chloride, rhodamineisothiocyanate, and Texas Red. Photolabels include, but are not limited to, rare earth metal cryptates, europium trisbipyridine diamine, a europium cryptat or chelate, Tb tribipyridine, diamine, dicyanins, La Jolla blue dye, allophycocyanin, phycocyanin B, phycocyanin C, phycocyanin R, thiamine, phycoerythrocyanin, phycoerythrin R, an up-converting or down-converting phosphor, luciferin, or acridinium esters. A variety of other known fluorescent or photolabels may be utilized. (See, e.g., U.S. Pat. No. 5,800,992; U.S. Pat. No. 6,319,668.)

For example, nanoparticle labeled nucleotides may be used. The nanoparticles may be silver or gold nanoparticles, although any nanoparticles capable of providing a detectable signal may be used. Nanoparticles of between 1 nm and 3 nm in diameter may be used, although nanoparticles of different dimensions and mass are contemplated. Methods of preparing nanoparticles are known. (See e.g., U.S. Pat. Nos. 6,054,495; 6,127,120; 6,149,868; Lee and Meisel, J. Phys. Chem. 86:3391-3395, 1982.) Nanoparticles may also be obtained from commercial sources (e.g., Nanoprobes Inc., Yaphank, N.Y.; Polysciences, Inc., Warrington, Pa.). Modified nanoparticles are available commercially, such as Nanogold® nanoparticles from Nanoprobes, Inc. (Yaphank, N.Y.). Nanogold® nanoparticles may be obtained with either single or multiple maleimide, amine or other groups attached per nanoparticle. The Nanogold® nanoparticles also are available in either positively or negatively charged form. Such modified nanoparticles may be covalently attached to nucleotides either before or after the nucleotides are incorporated into nucleic acids. Nanoparticles or other labels may be attached to nucleotides via any known linker compound to reduce steric hindrance and facilitate nucleic acid polymerization.

Labeled nucleotides may be incorporated into complementary nucleic acid strands made from a nucleic acid template. Alternatively, labels, may be attached to a particular type of nucleotide after synthesis of the nucleic acid. In other alternatives, the label may be attached by antibody-antigen interactions after nucleic acid synthesis. A label may be attached to one end of a nucleic acid molecule, such as the 5′ or the 3′ end, to serve as a unique tag for the end of the molecule. A modified nucleic acid with a unique tag may be used, for example, as a starting point for distance maps, with distances determined from the 5′ or 3′ end of the molecule. As one non-limiting example, a fluorescein or biotin label may be attached to the 5′ end of the nucleic acid (U.S. Pat. No. 6,344,316).

Nucleic Acids

Nucleic acids to be sequenced may be prepared by any technique known in the art. For example, the nucleic acid molecules may be naturally occurring DNA or RNA molecules, such as chromosomal DNA or messenger RNA (mRNA). Virtually any naturally occurring nucleic acid molecules may be prepared and analyzed by the disclosed methods including, without limit, chromosomal, mitochondrial or chloroplast DNA or ribosomal, transfer, heterogeneous nuclear or messenger RNA. Methods for preparing and isolating various forms of cellular nucleic acids are known. (See, e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989.) Generally, cells, tissues or other source material containing nucleic acids to be sequenced are first homogenized, for example by freezing in liquid nitrogen followed by grinding in a morter and pestle. Certain tissues may be homogenized using a Waring blender, Virtis homogenizer, Dounce homogenizer or other homogenizer. Crude homogenates may be extracted with detergents, such as sodium dodecyl sulphate (SDS), Triton X-100, CHAPS (3-[(3-cholamidopropyl)-dimethylammonio]-1-propane sulfonate), octylglucoside or other detergents known in the art. Alternatively or in addition, extraction may use chaotrophic agents such as guanidinium isothiocyanate, or organic solvents such as phenol. Protease treatment, for example with proteinase K, may be used to degrade cell proteins. Particulate contaminants may be removed by centrifugation or ultracentrifugation (for example, 10 to 30 min at about 5,000 to 10,000×g, or 30 to 60 min at about 50,000 to 100,000×g). Dialysis against aqueous buffer of low ionic strength may be of use to remove salts or other soluble contaminants. Nucleic acids may be precipitated by addition of ethanol at −20° C., or by addition of sodium acetate (pH 6.5, about 0.3 M) and 0.8 volumes of 2-propanol. Precipitated nucleic acids may be collected by centrifugation or, for chromosomal DNA, by spooling the precipitated DNA on a glass pipet or other probe.

The skilled artisan will realize that the procedures listed above are exemplary only and that many variations may be used, depending on the particular type of nucleic acid to be sequenced. For example, mitochondrial DNA is often prepared by cesium chloride density gradient centrifugation, using step gradients, while mRNA is often prepared using preparative columns from commercial sources, such as Promega (Madison, Wis.) or Clontech (Palo Alto, Calif.). Such variations are known in the art.

In cases where single stranded DNA (ssDNA) is to be analyzed, an ssDNA may be prepared from double stranded DNA (dsDNA) by any known method. Such methods may involve heating dsDNA and allowing the strands to separate, or may alternatively involve preparation of ssDNA from dsDNA by known amplification or replication methods, such as cloning into M13. Any such known method may be used to prepare ssDNA or ssRNA.

Although the discussion above concerns preparation of naturally occurring nucleic acids, virtually any type of nucleic acid could be analyzed by the disclosed methods. For example, nucleic acids prepared by various amplification techniques, such as polymerase chain reaction (PCR™) amplification, could be analyzed. (See U.S. Pat. Nos. 4,683,195, 4,683,202 and 4,800,159.) Nucleic acids to be analyzed may be cloned in standard vectors, such as plasmids, cosmids, BACs (bacterial artificial chromosomes) or YACs (yeast artificial chromosomes). (See, e.g., Berger and Kimmel, 1987; Sambrook et al., 1989.) Nucleic acid inserts may be isolated from vector DNA, for example, by excision with appropriate restriction endonucleases, followed by agarose gel electrophoresis. Methods for isolation of insert nucleic acids are well known.

Nucleic acids to be analyzed may be isolated from a wide variety of organisms including, but not limited to, viruses, bacteria, pathogenic organisms, eukaryotes, plants, animals, mammals, dogs, cats, sheep, cattle, swine, goats and humans. Also contemplated for use are amplified nucleic acids or amplified portions of nucleic acids.

Isolation of Single Nucleic Acid Molecules

Nucleic acids to be analyzed may be a single molecule of ssDNA or ssRNA. A variety of methods for selection and manipulation of single ssDNA or ssRNA molecules may be used, for example, hydrodynamic focusing, micro-manipulator coupling, optical trapping, or combination of these and similar methods. (See, e.g., Goodwin et al., 1996, Acc. Chem. Res. 29:607-619; U.S. Pat. Nos. 4,962,037; 5,405,747; 5,776,674; 6,136,543; 6,225,068.)

Microfluidics or nanofluidics may be used to sort and isolate template nucleic acids. Hydrodynamics may be used to manipulate nucleic acids into a microchannel, microcapillary, or a micropore. Hydrodynamic forces may be used to move nucleic acid molecules across a comb structure to separate single nucleic acid molecules. Once the nucleic acid molecules have been separated, hydrodynamic focusing may be used to position the molecules. A thermal or electric potential, pressure or vacuum may also be used to provide a motive force for manipulation of nucleic acids. Manipulation of template nucleic acids for analysis may involve the use of a channel block design incorporating microfabricated channels and an integrated gel material, as disclosed in U.S. Pat. Nos. 5,867,266 and 6,214,246.

Alternatively, a sample containing a nucleic acid template may be diluted prior to coupling to an immobilization surface. The immobilization surface may be in the form of magnetic or non-magnetic beads or other discrete structural units. At an appropriate dilution, each bead will have a statistical probability of binding zero or one nucleic acid molecules. Beads with one attached nucleic acid molecule may be identified using, for example, fluorescent dyes and flow cytometer sorting or magnetic sorting. Depending on the relative sizes and uniformity of the beads and the nucleic acids, it may be possible to use a magnetic filter and mass separation to separate beads containing a single bound nucleic acid molecule. In other alternatives, multiple nucleic acids attached to a single bead or other immobilization surface may be sequenced.

In further alternatives, a coated fiber tip may be used to generate single molecule nucleic acid templates for sequencing (e.g., U.S. Pat. No. 6,225,068). The immobilization surfaces may be prepared to contain a single molecule of avidin or other cross-linking agent. Such a surface could attach a single biotinylated primer, which in turn can hybridize with a single template nucleic acid to be sequenced. This is not limited to the avidin-biotin binding system, but may be adapted to any coupling system known in the art.

In other alternatives, an optical trap may be used for manipulation of single molecule nucleic acid templates for sequencing. (E.g., U.S. Pat. No. 5,776,674). Exemplary optical trapping systems are commercially available from Cell Robotics, Inc. (Albuquerque, N. Mex.), S+L GmbH (Heidelberg, Germany) and P.A.L.M. Gmbh (Wolfratshausen, Germany).

Nucleic Acid Synthesis

Certain methods may involve synthesis of a complementary nucleic acid sequence from a template, for example by use of a DNA or RNA polymerase. Non-limiting examples of polymerases of potential use include DNA polymerases, RNA polymerases, reverse transcriptases, and RNA-dependent RNA polymerases. The differences between these polymerases in terms of their “proofreading” activity and requirement or lack of requirement for primers and promoter sequences are known in the art. Where RNA polymerases are used as the polymerase, the template molecule to be sequenced may be double-stranded DNA. Methods of using polymerases to synthesize nucleic acids from labeled nucleotides, are known. (See, e.g., U.S. Pat. Nos. 4,962,037; 5,405,747; 6,136,543; 6,210,896.)

Polymerase

Non-limiting examples of polymerases that may be used include Thermatoga maritima DNA polymerase, AmplitaqFS™ DNA polymerase, Taquenase™ DNA polymerase, ThermoSequenase™, Taq DNA polymerase, Qbeta™ replicase, T4 DNA polymerase, Thermus thermophilus DNA polymerase, RNA-dependent RNA polymerase and SP6 RNA polymerase.

A number of polymerases are commercially available, including Pwo DNA Polymerase from Boehringer Mannheim Biochemicals (Indianapolis, Ind.); Bst Polymerase from Bio-Rad Laboratories (Hercules, Calif.); IsoTherm™ DNA Polymerase from Epicentre Technologies (Madison, Wis.); Moloney Murine Leukemia Virus Reverse Transcriptase, Pfu DNA Polymerase, Avian Myeloblastosis Virus Reverse Transcriptase, Thermus flavus (Tfl) DNA Polymerase and Thermococcus litoralis (Tli) DNA Polymerase from Promega (Madison, Wis.); RAV2 Reverse Transcriptase, HIV-1 Reverse Transcriptase, T7 RNA Polymerase, T3 RNA Polymerase, SP6 RNA Polymerase, RNA Polymerase E. coli, Thermus aquaticus DNA Polymerase, T7 DNA Polymerase ±3′→5′ exonuclease, Klenow Fragment of DNA Polymerase I, Thermus ‘ubiquitous’ DNA Polymerase, and DNA polymerase I from Amersham Pharmacia Biotech (Piscataway, N.J.). However, any polymerase that is known in the art for the template dependent polymerization of nucleotides may be used. (See, e.g., Goodman and Tippin, Nat. Rev. Mol. Cell Biol. 1(2):101-9, 2000; U.S. Pat. No. 6,090,589.)

Various methods are known for adjusting the rate of polymerase activity, including adjusting the temperature, pressure, pH, salt concentration, divalent cation concentration, or the concentration of nucleotides in the reaction chamber. Methods of optimization of polymerase activity are known to the person of ordinary skill in the art.

Primers

Generally, primers are between ten and twenty bases in length, although longer primers may be employed. Primers may be designed to be exactly complementary in sequence to a known portion of a template nucleic acid molecule. Known primer sequences may be used, for example, where primers are selected for identifying sequence variants adjacent to known constant chromosomal sequences, where an unknown nucleic acid sequence is inserted into a vector of known sequence, or where a native nucleic acid has been sequenced partially. Methods for synthesis of primer of any sequence are known and automated oligonucleotide synthesizers are commercially available See, e.g., Applied Biosystems, Foster City, Calif.; Millipore Corp., Bedford, Mass.

Alternatively, a nucleic acid may be synthesized in the absence of a known primer-binding site. In such cases, it may be possible to use random primers, such as random hexamers or random oligomers of 7, 8, 9, 10, 11, 12, 13, 14, 15 bases or greater length, to initiate polymerization.

Where nucleic acids are to be sequenced, custom-designed software packages may be used to analyze the data. Data analysis may be performed, for example, using a computer and publicly available software packages. Non-limiting examples of available software for DNA sequence analysis include the PRISM™ DNA Sequencing Analysis Software (Applied Biosystems, Foster City, Calif.), the Sequencher™ package (Gene Codes, Ann Arbor, Mich.), and a variety of software packages available through the National Biotechnology Information Facility website.

Nucleic Acid Amplification

A number of template dependent processes are available to amplify template nucleic acids to be analyzed. One of the best known amplification methods is polymerase chain reaction (PCR) (U.S. Pat. Nos. 4,683,195; 4,683,202; 4,800,159; Innis et al., PCR Protocols, Academic Press, Inc., San Diego Calif., 1990).

A reverse transcriptase PCR amplification procedure may be performed. Methods of reverse transcribing RNA into cDNA are well known (e.g., Sambrook et al., 1989). Alternative methods for reverse transcription utilize thermostable DNA polymerases. Polymerase chain reaction methodologies are well known in the art.

Another method for amplification is ligase chain reaction (“LCR”), disclosed in European Application No. 320 308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs.

Qbeta Replicase (PCT Application No. PCT/US87/00880) may also be used as another amplification method. In this method, a replicative sequence of RNA that has a region complementary to that of a target is added to a sample in the presence of an RNA polymerase. The polymerase will copy the replicative sequence.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids (Walker et al., Proc. Nat'l Acad. Sci. USA 89:392-396, 1992).

Strand Displacement Amplification (SDA) is another method of carrying out isothermal amplification of nucleic acids, involving multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for attachment of labels. A similar approach is used in SDA.

Still other amplification methods are disclosed in GB Application No. 2 202 328. Modified primers are used in a PCR like, template and enzyme dependent synthesis. The primers may be modified by labeling with a capture moiety (e.g., biotin) and/or a detector moiety (e.g., enzyme).

Other nucleic acid amplification procedures include transcription-based amplification systems (TAS), including nucleic acid sequence based amplification (NASBA) and 3SR. (Kwoh et al., Proc. Nat'l Acad. Sci. USA 86:1173, 1989; Gingeras et al., PCT Application WO 88/10315). In NASBA, the nucleic acids may be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, RNA is reverse transcribed into double stranded DNA, and transcribed once against with a polymerase such as T7 or SP6.

Davey et al. (European Application No. 329 822) disclose a nucleic acid amplification process involving cyclically synthesizing single-stranded RNA (“ssRNA”), ssDNA, and double-stranded DNA (dsDNA). The ssRNA is a first template for a first primer oligonucleotide, which is elongated by reverse transcriptase (RNA-dependent DNA polymerase). The RNA is then removed from the resulting DNA:RNA duplex by the action of ribonuclease H (RNase H). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle.

Miller et al. (PCT Application WO 89/06700) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR.” (Frohman, M. A., In: PCR PROTOCOLS: A GUIDE TO METHODS AND APPLICATIONS, Academic Press, N.Y., 1990; Ohara et al., Proc. Nat'l Acad. Sci. USA, 86:5673-5677, 1989).

Following amplification, it may be appropriate to separate the amplification product from the template and excess primer prior to analysis. Amplification products may be separated, for example, by agarose, agarose-acrylamide or polyacrylamide gel electrophoresis using standard methods (Sambrook et al., 1989).

Alternatively, chromatographic techniques may be employed to effect separation. There are many kinds of chromatography which may be used, such as affinity, adsorption, partition, ion-exchange and molecular sieve, and many specialized techniques for using them including column, paper, thin-layer and gas chromatography (Freifelder, Physical Biochemistry Applications to Biochemistry and Molecular Biology, 2nd ed., Wm. Freeman and Co., New York, N.Y., 1982).

Nucleic Acid Labeling

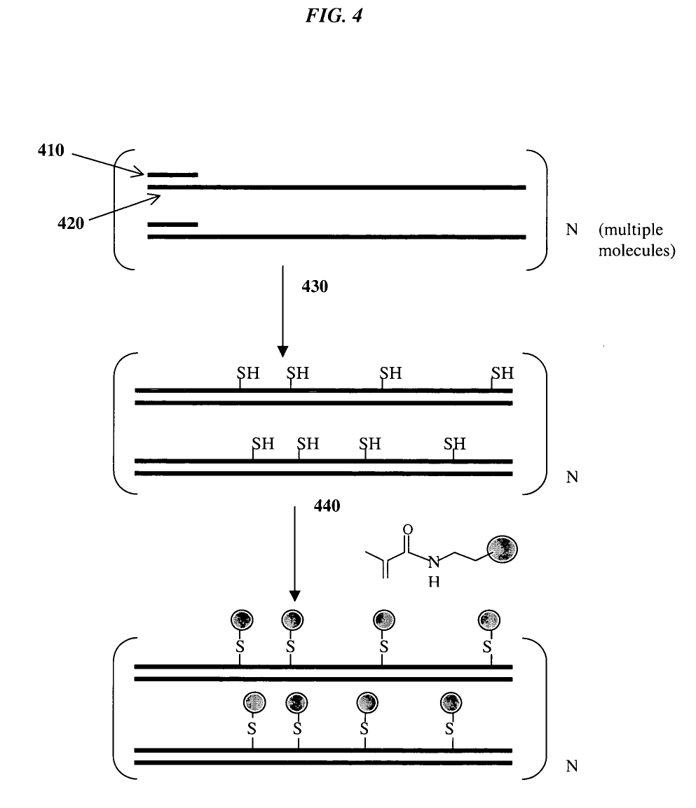

FIG. 4 illustrates exemplary methods for labeling nucleic acids with tag moieties. As indicated, multiple copies of a template nucleic acid strand 420 are allowed to anneal to a sequence specific primer 410. The primer 410 may be selected to bind to the 3′ end of the template strand 420, or to any selected internal site on the template 420. Methods for selection and synthesis of primers 410 are well known in the art (e.g., Frohman et al., 1990; Ohara et al., 1989; Sambrook et al., 1989). The primer-template complex 410, 420 is incubated with DNA polymerase (e.g., Klenow fragment of DNA Polymerase I) in an appropriate buffer solution. Kits comprising DNA polymerase with appropriate 10× buffer solutions are commercially available from many sources.

A mixture 430 containing all four deoxynucleoside triphosphates is added. Where, for example, adenosine residues are to be labeled, the mixture 430 contains unlabeled deoxycytidine triphosphate (dCTP), deoxyguanosine triphosphate (dGTP) and deoxythymidine triphosphate (dTTP). The mixture 430 also contains a combination of derivatized and underivatized deoxyadenosine triphosphate (dATP). The ratios of derivatized and underivatized nucleotides may vary, depending upon the relative abundance of labeled nucleotides to be incorporated into the complementary nucleic acid. However, a proportion of labeled nucleotide of about 1, 2, 5, 10, 15, 20, 25, 30, 35, 40, 45 or 50% is contemplated. In a specific non-limiting example, the mixture 430 contains 10% thiol-modified dATP and 90% unmodified dATP.

The polymerase synthesizes a multiplicity of complementary strands, each of which contains about 10% thiol-modified adenosine residues. The sulfhydryl groups may be used to covalently attach a label moiety 440 to the complementary strand. For example, an acrydite group attached to gold nanoparticles 440 could be covalently linked to the sulfhydryl residues on the complementary nucleic acids. The nanoparticles 440 would end up attached to the complementary strand, in locations where adenosine residues are present. Because multiple labeled complementary strands may be analyzed, a distance map indicating the locations of each adenosine residue in the complementary sequence may be compiled. The process may be repeated for each of the four types of nucleotides to obtain distance maps for each nucleotide type. The four maps may be integrated by known methods to obtain the complete sequence of the complementary strand. The template strand 420 sequence may be determined from the sequence of the complementary strand. As indicated above, nucleic acids may be identified using less than all four nucleotide maps. Further, by sequencing both strands of double-stranded DNA, a complete nucleic acid sequence may be obtained by labeling only two of the four bases (e.g., only purines or only pyrimnidines).

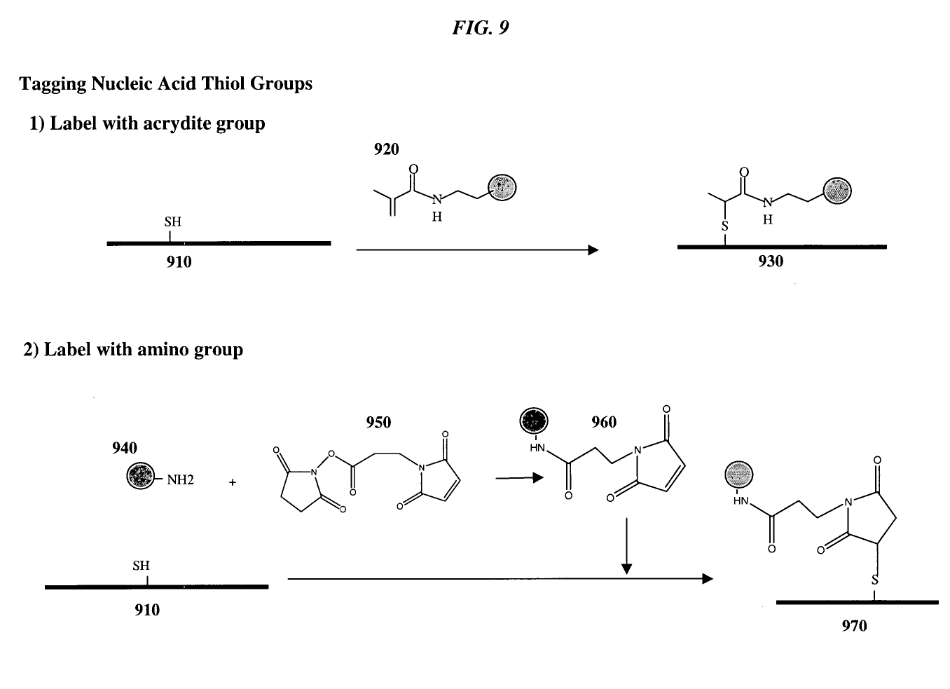

The skilled artisan will realize that thiol modified nucleotides are only one example of nucleotide derivatives that may be incorporated into a nucleic acid and that many other types of derivatized nucleotides may be incorporated into a nucleic acid within the scope of the claimed methods. For example, nucleotides modified with functional groups such as sulfhydryl, amine and carboxyl moieties may all be incorporated into nucleic acids using known methods, for example using alkynylamino-nucleotide chemistry (e.g., U.S. Pat. No. 5,151,507). Each type of modified nucleotide may be attached to one or more types of labels after incorporation of the nucleotide into a nucleic acid. Exemplary methods for attachment of labels to derivatized nucleic acids are further disclosed in FIG. 9 through FIG. 11.

FIG. 9 illustrates exemplary methods for labeling nucleic acids 910 containing sulfhydryl modified nucleotides. Thiol-containing nucleic acids 910 are prepared from sulfhydryl modified nucleotides as disclosed in U.S. Pat. No. 5,151,507. In one method, a label 920 comprising an acrydite group is reacted with the nucleic acid 910 using standard chemistries to form an acrydite labeled nucleic acid 930. In an alternative method, a label 940 containing an amine residue is activated, for example with N-succinimidyl-4-maleimidobutyrate 950 to form an activated label complex 960. The activated label 960 is then reacted with a thiol-containing nucleic acid 910 to create a labeled nucleic acid 970.

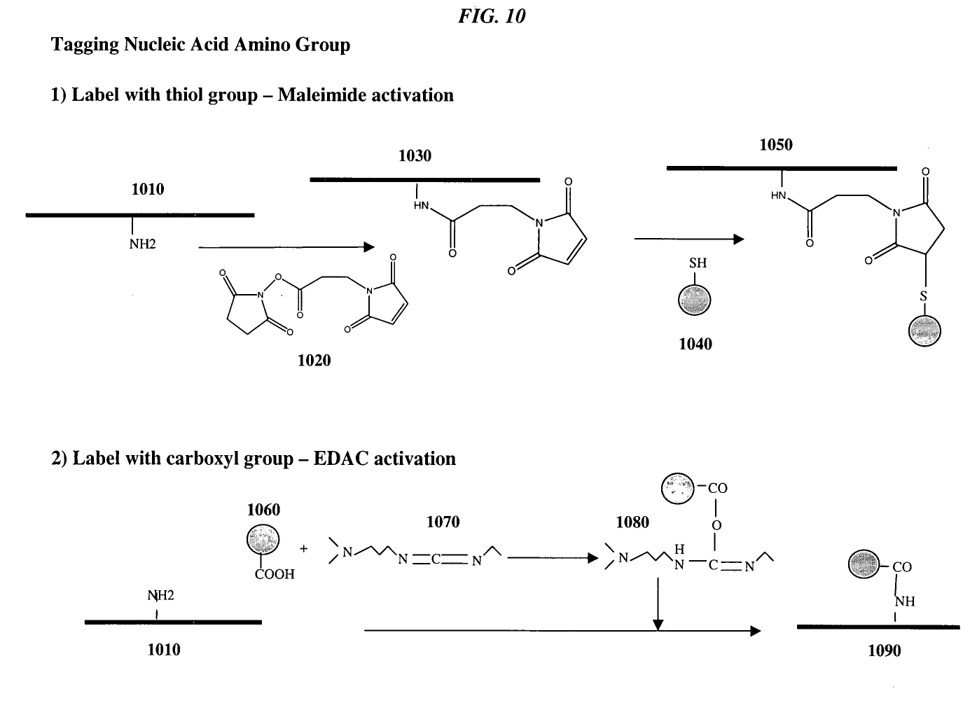

FIG. 10 illustrates exemplary methods for labeling nucleic acids 1010 containing amine modified nucleotides. In one method, the amine moiety is first activated with N-succinimidyl-4-maleimidobutyrate 1020 to form an activated nucleic acid 1030. The activated nucleic acid 1030 is reacted with a sulfhydryl-containing label 1040 to form a labeled nucleic acid 1050. In an alternative method, a label 1060 containing a carboxyl moiety is activated with a water soluble carbodiimide 1070, such as EDAC (1-ethyl-3-(3-dimethylaminopropyl)carbodiimide) to form a reactive label complex 1080. The reactive label 1080 then reacts with the amine moiety on the nucleic acid 1010, with elimination of the carbodiimide residue 1070, to form a labeled nucleotide 1090. The EDAC 1070 catalyzed formation of covalent bonds between amino and carboxyl groups is similar to the chemistry of solid phase peptide synthesis, the protocols for which are well known in the art.

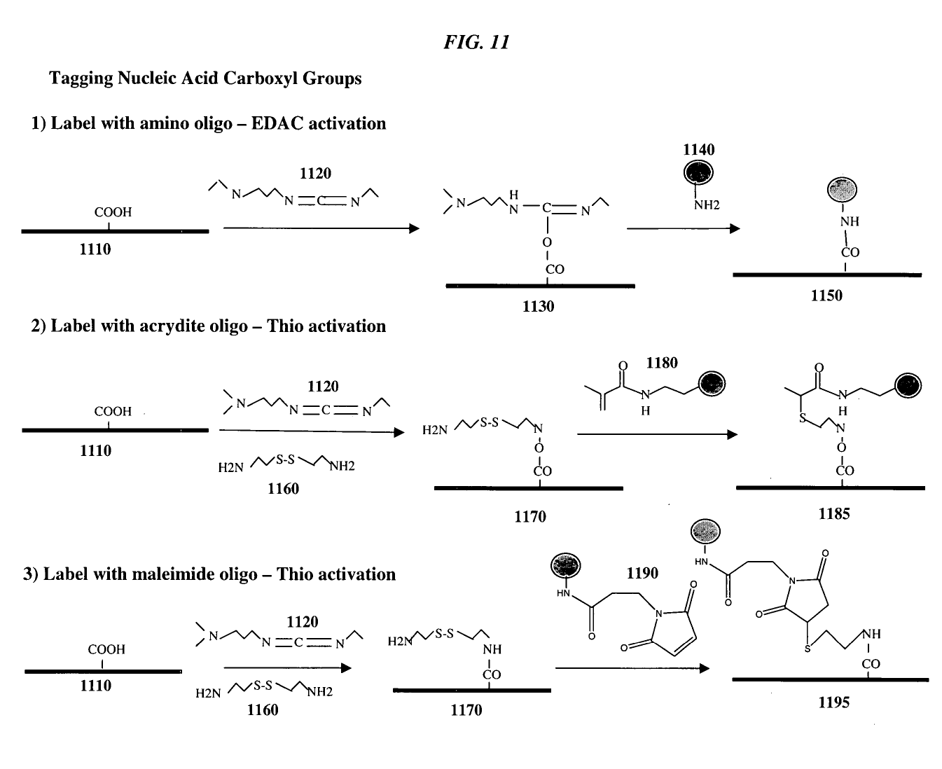

FIG. 11 shows exemplary methods for labeling nucleic acids 1110 containing carboxyl modified nucleotides. In one method, a nucleic acid 1110 containing carboxyl moieties is first activated with a carbodiimide 1120 to form an activated nucleic acid 1130. The activated intermediate is reacted with an amine label 1140, resulting in elimination of the carbodiimide 1120 moiety and formation of a covalently labeled nucleic acid 1150. In another method, a nucleic acid 1110 is reacted with a carbodiimide 1120 in the present of cystamine 1160. This results in formation of an activated nucleic acid 1170 containing a disulfide group. The disulfide is then reacted with an acrydite label 1180 to form a covalently labeled nucleic acid 1185. In another alternative method, the disulfide containing nucleic acid 1170 is reacted with a maleimide label 1190 to form a labeled nucleic acid 1195. These and many other labeling methods are known in the art and any such known method may be used.

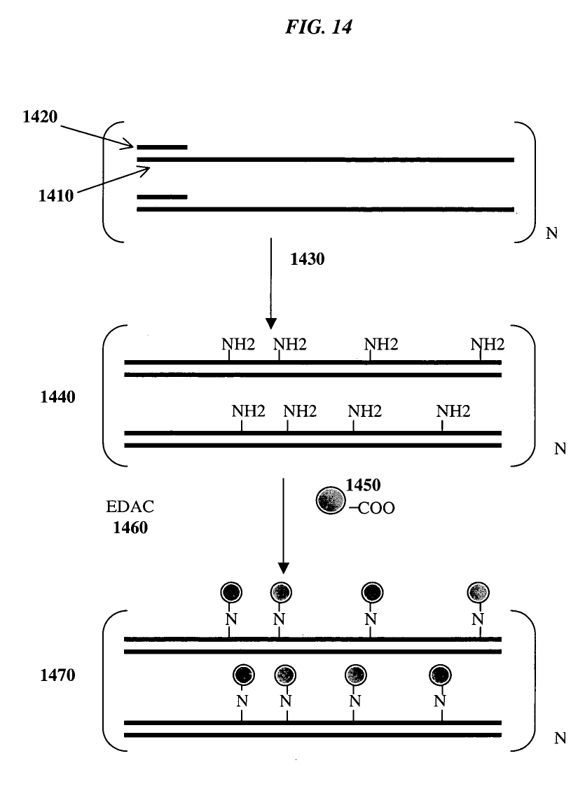

Alternative methods for tagging nucleic acids are illustrated in FIG. 14. A set of “N” template nucleic acids 1410 may be hybridized to complementary primers 1420. Using a DNA polymerase in buffer solution with a mixture of deoxynucleotide triphosphates, complementary strands 1440 may be synthesized. In a non-limiting example, the solution may contain 10% amine-derivatized deoxyadenosine triphosphate (dATP) and 90% non-derivatized dATP, resulting in the random incorporation of amine-derivatized nucleotides in the complementary strands 1440. Amine-derivatized dATP may be made by methods similar to those disclosed in U.S. Pat. No. 5,151,507. The amine-derivatized nucleotides may be attached to, for example, a carboxyl label 1450, using activation by a water-soluble carbodiimide 1460. The carbodiimide activation results in formation of multiple strands of labeled nucleic acids 1470, that may be analyzed to produce distance maps. As discussed above, in certain alternative methods only one type of nucleotide, two types of nucleotides or all four types of nucleotides may be labeled and analyzed.

### EXAMPLES

### Example 1

### Nucleic Acid Identification and Sequencing

Sensor Layer Construction

Photolithography is used to create an array of multiplaner structures (0.5×0.5 μm) on a silicon substrate, each structure with a silicon base support and two or more layers of a light opaque material interspersed with one or more layers of a light translucent material. The light opaque layers are formed of a thin layer of chrome, although silver, gold or other light opaque metals may be used. The light translucent layers are formed of silicon, although any material that is relatively translucent at the wavelengths of light emitted by a photolabel may be used, such as glass or certain types of plastics. Polymethylmethacrylate is coated on the chip to enhance signal detection. Alternative polymeric materials that may be used include polymethylmethacrylate, ultraviolet-curable polyurethanes and epoxies, and other polymers that exhibit optical transparency and low fluorescence at excitation wavelengths. The polymeric material is formed into appropriate structures by polymer casting and chemical curing (Kim et al., Nature 376: 581-584 1995).

A chip containing the multiplanar structures is divided into two parts. A layer of resist is coated on the sides of each chip part, perpendicular to the opaque and translucent layers. An AFM/STP tip is used to etch 10 nm lines in the resist layer overlaying each structure. Chemical etching is used to create nano-scale grooves in each of the structures. When the chip parts are aligned and fused together, the grooves form nanochannels, which extend through the sensor layers. The chip containing the photosensor layers is inserted into an apparatus comprising first and second buffer chambers. Electrodes are attached to the first and second chambers, negative in the first chamber and positive in the second chamber.

Preparation of Labeled Nucleic Acids

Target DNA to be analyzed is purified from a sample of tissues, cultured cells or any other source of nucleic acid by known methods (e.g. Sambrook et al., 1989). The DNA (1-10 μg) is digested with a restriction endonuclease (e.g., Bam HI) for 1 hour at 37° C., using manufacturer supplied buffer (e.g., New England Biolabs). A target DNA fragment is purified by electrophoresis in low melting point agarose (e.g., Sambrook et al., 1989) and used as a template for polymerase incorporation of derivatized nucleotides into a complementary strand.

Alternatively, PCR amplification may be used to amplify and isolate a DNA fragment for analysis. Where at least part of the target sequence is known (for example, analysis of single nucleotide polymorphism sites), PCR primers may be designed to amplify the selected target sequence. Primers of any designated sequence may be obtained from a wide variety of commercial sources well known in the art. The primers are used to amplify the target DNA sequence using, for example, Taq polymerase (Roche Applied Sciences). Where the target sequence is not know, oligonucleotide adaptors (synthetic double-stranded DNA fragments containing a selected restriction enzyme site, e.g. Bam HI) may be obtained from commercial sources and ligated to the digested and purified DNA fragment of interest. After removal of excess adaptors, the target DNA is amplified by PCR. Amplified DNA is purified, for example, using a commercial DNA purification kit (e.g., Qiagen). The amplified nucleic acid may serve as a template for polymerase incorporation of derivatized nucleotides into a complementary strand.

In another alternative, human chromosomal DNA is purified according to Sambrook et al. (1989). Following digestion with Bam HI, the genomic DNA fragments are inserted into the multiple cloning site of the pBluescript® II phagemid vector (Stratagene, Inc., La Jolla, Calif.) and grown up in E. coli. After plating on ampicillin-containing agarose plates a single colony is selected and grown up for analysis. Single-stranded DNA copies of the genomic DNA insert are rescued by co-infection with helper phage. After digestion in a solution of proteinase K:sodium dodecyl sulphate (SDS), the DNA is phenol extracted and then precipitated by addition of sodium acetate (pH 6.5, about 0.3 M) and 0.8 volumes of 2-propanol. The DNA containing pellet is resuspended in Tris-EDTA buffer and stored at −20° C. until use. Agarose gel electrophoresis shows a single band of purified DNA.

M13 forward primers complementary to the known pBluescript® sequence, located next to the genomic DNA insert, are purchased from Midland Certified Reagent Company (Midland, Tex.). The primers are covalently modified to contain a biotin moiety attached to the 5′ end of the oligonucleotide. The biotin group is covalently linked to the 5′-phosphate of the primer via a (CH₂)₆ spacer. Biotin-labeled primers are allowed to hybridize to the ssDNA template molecules prepared from the pBluescript® vector. The primer-template complexes are then attached to streptavidin-coated beads according to Dorre et al. (Bioimaging 5: 139-152, 1997).

The primer-template is incubated with modified T7 DNA polymerase (United States Biochemical Corp., Cleveland, Ohio). Labeled nucleic acids are prepared by incorporation of amine-derivatized nucleotides into a complementary strand. Four sets of labeled nucleic acids are prepared, each labeled on a different type of nucleotide. Each set of labeled nucleic acid is prepared using a mixture of 10% amine-derivatized nucleotide and 90% underivatized nucleotide. The labeling process results in a multiplicity of labeled nucleic acids being formed, each one containing about 10% labeling for a particular type of nucleotide, e.g., adenosine, with the labels located on different sets of adenosine residues in each labeled nucleic acid. The template strands are separated by heating and washing of the streptavidin-coated beads. After removal of the unlabeled template strand, the derivatized complementary strand is labeled. The amine-derivatized nucleotides are reacted with mono-sulfo-NHS Nanogold® particles (Nanoprobes, Yaphank, N.Y.), which are 1.4 nm Nanogold® particles containing a reactive sulfo-N-hydroxysuccinimide ester that reacts with primary amines. The labeling protocol follows manufacturer's instructions. Briefly, lyophilized nanoparticles are rehydrated in 1 ml of distilled water. The rehydrate contains 0.02 mM Hepes, pH 7.5. Amine-derivatized nucleic acids are incubated with the derivatized nanoparticles for 1 hour at room temperature. Labeled nucleic acids tagged with gold nanoparticles are formed. The labeled nucleic acids are separated from unlabeled nucleic acids and unreacted nanoparticles by gel permeation chromatography on Superose 6 (Pharmacia), eluted with 0.02M sodium phosphate (pH 7.4). Eluted labeled nucleic acids are concentrated by centrifugation on a Centricon-30 filter (Amicon). The nanoparticle-labeled nucleic acids are analyzed by measuring conductivity detection, using nanopores operably coupled to electrical detectors.

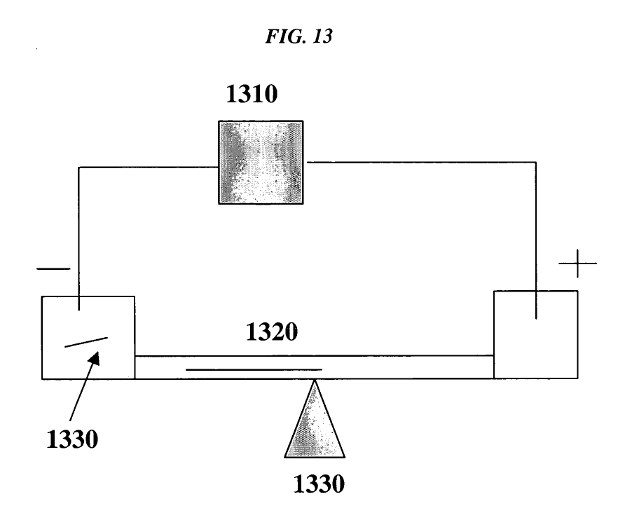

FIG. 13 illustrates an exemplary apparatus for electrical detection of nanoparticle-labeled nucleic acids 1330. A power supply 1310 is connected to electrodes at opposite ends of a nanochannel 1320. Labeled nucleic acids 1330 are placed into a chamber at one end of the nanochannel 1320. The nanochannel is of a diameter (e.g., about 5 nm) to allow only one nucleic acid 1330 to pass at a time. In response to an imposed electrical gradient, negative in the chamber and positive at the other end of the nanochannel 1320, nucleic acids pass through the nanochannel past a detector 1330. In one non-limiting example, the detector 1330 is an electrical detector that measures the conductance across the nanochannel. As nanoparticle labeled nucleic acids 1330 pass the detector 1330, the nanoparticle tags are detected by the change in conductance.

In an alternative method, labeled nucleic acids are prepared by incorporation of thiol-derivatized nucleotides into a complementary strand. Two different protocols are followed. In the first protocol, four sets of labeled nucleic acids are prepared, each labeled on a different type of nucleotide. Each set of labeled nucleic acid is prepared using a mixture of 10% thiol-derivatized nucleotide and 90% underivatized nucleotide. As discussed above, each of the four types of nucleotide is labeled in a separate batch. Thus, for example, the adenosine-labeled batch contains 10% thiol-derivatized adenosine and 90% underivatized adenosine, with 100% underivatized nucleotide for the guanosine, cytidine and thymidine nucleotides. The labeling process results in a multiplicity of labeled nucleic acids being formed, each one containing about 10% labeling for a particular type of nucleotide, e.g., adenosine, with the labels generally located on different sets of adenosine residues in each labeled nucleic acid. In a second protocol, only one set of labeled nucleic acids is prepared, labeled on a single type of nucleotide (for example, adenosine). The template strands are separated by heating and washing of the streptavidin-coated beads. After removal of the unlabeled template strand, the derivatized complementary strand is labeled.

Thiol-derivatized nucleic acids are labeled with a fluorescein Raman label. The label is added as fluorescein-5-maleimide (F-150, Molecular Probes, Eugene, Oreg.). Covalent reaction of the maleimide moiety with the nucleic acid sulfhydryl groups is performed according to the manufacturer's instructions. A 50 μM solution of thiolated nucleic acid is dissolved in 100 mM phosphate buffered saline (PBS, pH 7.4) at room temperature. A 10 mM stock solution of fluorescein maleimide is freshly prepared and stored in a light-opaque container. A 10:1 molar excess of fluorescein maleimide to nucleic acid sulfhydryl groups is added to the nucleic acid. The reagent is added drop wise with stirring. The reaction is allowed to proceed for 2 hours at room temperature in the dark. Upon completion, a molar excess of mercaptoethanol is added to react with any remaining reagent. The fluorescein-conjugated nucleic acid is separated by washing of the streptavidin coated beads. The fluorescein-labeled nucleic acids are removed from the beads and added to the first buffer chamber of a nucleic acid sequencing apparatus for Raman detection and construction of distance maps. The biotin moiety at the 5′ end of the nucleic acid serves as a starting point for distance map construction.

Raman Detection

Fluorescein-labeled nucleic acids are added to the first buffer chamber of a sequencing apparatus in 10 mM phosphate buffer, pH 7.4. Where all four types of nucleotides are labeled, each of the four labeled batches is placed into a separate first buffer chamber, each chamber operably coupled to a different sensor layer and second buffer chamber. A 100 volt electrical potential is induced between the first and second buffer chambers, negative on top and positive on the bottom. In response to the electrical potential, the negatively charged nucleic acids pass from the first buffer chamber, through nanochannels in the sensor layers and into the second buffer chamber.

The fluorescein moieties on the labeled nucleic acids are excited by laser illumination, as discussed in more detail in Example 2. Excitatory light passes through a transparent window in the first chamber, located immediately above the sensor layers. The labeled nucleotides pass through the light opaque layer, cutting off the source of excitatory light and shielding the photodetector from the light source. As the excited label passes the photon-sensing layer, it emits a photon. The emitted photon is detected by a photodetector, according to Example 2. The detected signal is amplified by an amplifier and stored and processed by a computer. The computer also records the time at which each labeled nucleotide passes through the nanochannel, allowing the calculation of distances between adjacent labeled nucleotides and the compilation of a distance map for each type of labeled nucleotide. Where a single type of nucleotide is labeled, the distance map is used to identify the nucleic acid by the pattern of labeled residues. Where all four types of nucleotides are labeled, the four resulting distance maps are compiled to produce the sequence of the nucleic acid.

### Example 2

### Raman Detection of Nucleotides

Methods and Apparatus

In a non-limiting example, the excitation beam of a Raman detection unit was generated by a titanium:sapphire laser (Mira by Coherent) at a near-infrared wavelength (750-950 nm) or a gallium aluminum arsenide diode laser (PI-ECL series by Process Instruments) at 785 nm or 830 nm. Pulsed laser beams or continuous beams were used. The excitation beam was passed through a dichroic mirror (holographic notch filter by Kaiser Optical or a dichromatic interference filter by Chroma or Omega Optical) into a collinear geometry with the collected beam. The transmitted beam passed through a microscope objective (Nikon LU series), and was focused onto the Raman active substrate where target analytes (nucleotides or purine or pyrimidine bases) were located.

The Raman scattered light from the analytes was collected by the same microscope objective, and passed the dichroic mirror to the Raman detector. The Raman detector comprised a focusing lens, a spectrograph, and an array detector. The focusing lens focused the Raman scattered light through the entrance slit of the spectrograph. The spectrograph (Acton Research) comprised a grating that dispersed the light by its wavelength. The dispersed light was imaged onto an array detector (back-illuminated deep-depletion CCD camera by RoperScientific). The array detector was connected to a controller circuit, which was connected to a computer for data transfer and control of the detector function.

For surface-enhanced Raman spectroscopy (SERS), the Raman active substrate consisted of metallic nanoparticles or metal-coated nanostructures. Silver nanoparticles, ranging in size from 5 to 200 nm, was made by the method of Lee and Meisel (J. Phys. Chem., 86:3391, 1982). Alternatively, samples were placed on an aluminum substrate under the microscope objective. The Figures discussed below were collected in a stationary sample on the aluminum substrate. The number of molecules detected was determined by the optical collection volume of the illuminated sample. Detection sensitivity down to the single molecule level was demonstrated.

Single nucleotides may also be detected by SERS using a 100 μm or 200 μm microfluidic channel. Nucleotides may be delivered to a Raman active substrate through a microfluidic channel (between about 5 and 200 μm wide). Microfluidic channels may be made by molding polydimethylsiloxane (PDMS), using the technique disclosed in Anderson et al. (“Fabrication of topologically complex three-dimensional microfluidic systems in PDMS by rapid prototyping,” Anal. Chem. 72:3158-3164, 2000).

Where SERS was performed in the presence of silver nanoparticles, the nucleotide, purine or pyrimidine analyte was mixed with LiCl (90 μM final concentration) and nanoparticles (0.25 M final concentration silver atoms). SERS data were collected using room temperature analyte solutions.

Results

Nucleoside monophosphates, purine bases and pyrimidine bases were analyzed by SERS, using the system disclosed above. Table 1 shows the present detection limits for various analytes of interest.

TABLE 1
SERS Detection of Nucleoside Monophosphates,
Purines and Pyrimidines

| | | Number of |
|---|---|---|
| Analyte | Final Concentration | Molecules Detected |
| dAMP | 9 picomolar (pM) | ˜1 molecule |
| Adenine | 9 pM | ˜1 molecule |
| dGMP | 90 μM | 6 × 10⁶ |
| Guanine | 909 pM | 60 |
| dCMP | 909 μM | 6 × 10⁷ |
| Cyotosine | 90 nM | 6 × 10³ |
| dTMP | 9 μM | 6 × 10⁵ |
| Thymine | 90 nM | 6 × 10³ |

Conditions were optimized for adenine nucleotides only. LiCl (90 μM final concentration) was determined to provide optimal SERS detection of adenine nucleotides. Detection of other nucleotides may be facilitated by use of other alkali-metal halide salts, such as NaCl, KCl, RbCl or CsCl. The claimed methods are not limited by the electrolyte solution used, and it is contemplated that other types of electrolyte solutions, such as MgCl, CaCl, NaF, KBr, LiI, etc. may be of use. The skilled artisan will realize that electrolyte solutions that do not exhibit strong Raman signals will provide minimal interference with SERS detection of nucleotides. The results demonstrate that the Raman detection system and methods disclosed above were capable of detecting and identifying single molecules of nucleotides and purine bases. This is the first report of Raman detection of unlabeled nucleotides at the single nucleotide level.

### Example 3

### Raman Emission Spectra of Nucleotides, Purines and Pyrimidines

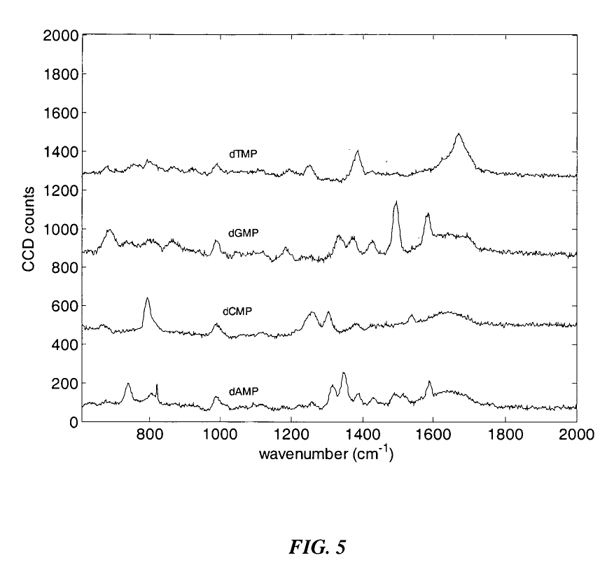

The Raman emission spectra of various analytes of interest were obtained using the protocol of Example 2, with the indicated modifications. FIG. 5 shows the Raman emission spectra of a 100 mM solution of each of the four nucleoside monophosphates, in the absence of surface enhancement and without Raman labels. No LiCl was added to the solution. A 10 second data collection time was used. Excitation occurred at 514 nm. Lower concentrations of nucleotides may be detected with longer collection times, added electrolytes and/or surface enhancement. For each of the following figures, a 785 nm excitation wavelength was used. As shown in FIG. 5, the unenhanced Raman spectra showed characteristic emission peaks for each of the four unlabeled nucleoside monophosphates.

Surface-enhanced Raman emission spectra were obtained for a 1 nM solution of guanine, a 100 nM solution of cytosine, and a 100 nm solution of thymine in the presence of LiCl and silver nanoparticles (not shown). Each spectrum exhibited characteristic peaks that may be used to identify and distinguish the four types of nucleotides (not shown).

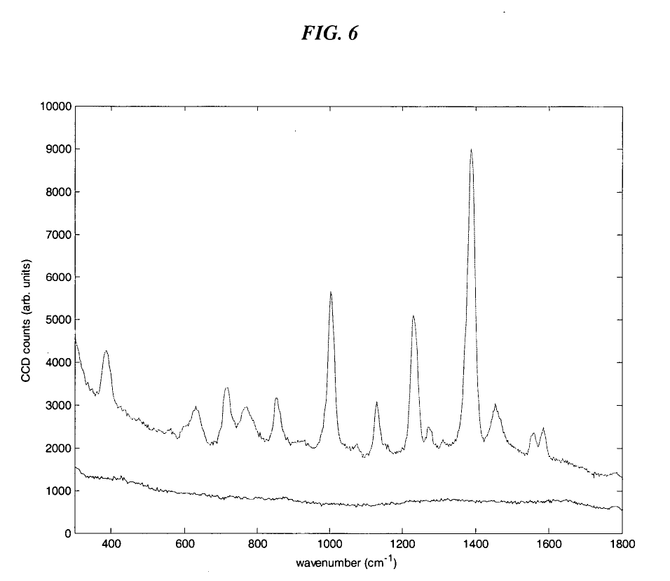

FIG. 6 shows the SERS spectrum of a 500 nM solution of dATP (lower trace) and fluorescein-labeled dATP (upper trace). dATP-fluorescein was purchased from Roche Applied Science (Indianapolis, Ind.). The Figure shows a strong increase in SERS signal due to labeling with fluorescein.

### Example 4

### SERS Detection of Nucleotides and Amplification Products

Silver Nanoparticle Formation

Silver nanoparticles used for SERS detection were produced according to Lee and Meisel (1982). Eighteen milligrams of AgNO₃ were dissolved in 100 mL (milliliters) of distilled water and heated to boiling. Ten mL of a 1% sodium citrate solution was added drop-wise to the AgNO₃ solution over a 10 min period. The solution was kept boiling for another hour. The resulting silver colloid solution was cooled and stored.

SERS Detection of Adenine

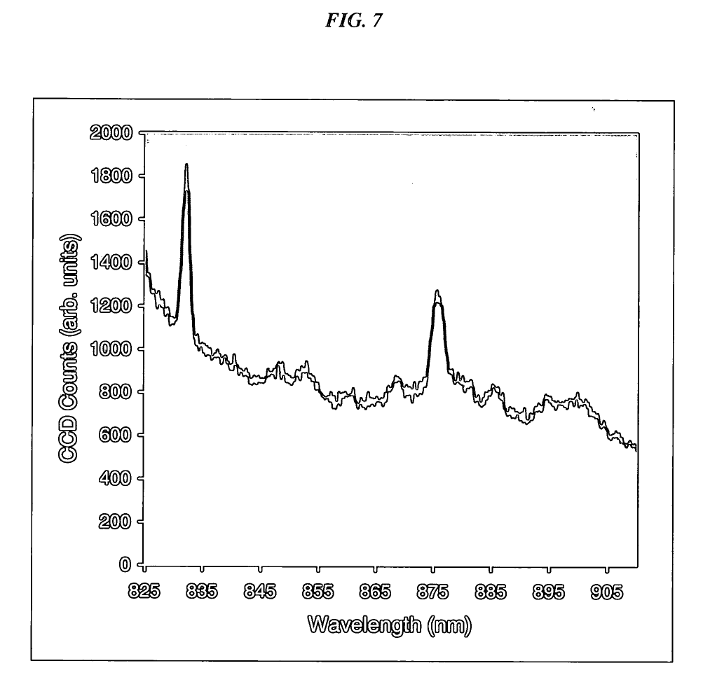

The Raman detection system was as disclosed in Example 2. One mL of silver colloid solution was diluted with 2 mL of distilled water. The diluted silver colloid solution (160 μL) (microliters) was mixed with 20 μL of a 10 nM (nanomolar) adenine solution and 40 μL of LiCl (0.5 molar) on an aluminum tray. The LiCl acted as a Raman enhancing agent for adenine. The final concentration of adenine in the sample was 0.9 nM, in a detection volume of about 100 to 150 femtoliters, containing an estimated 60 molecules of adenine. The Raman emission spectrum was collected using an excitation source at 785 nm excitation, with a 100 millisecond collection time. As shown in FIG. 7, this procedure demonstrated the detection of 60 molecules of adenine, with strong emission peaks detected at about 833 nm and 877 nm. As discussed in Example 2, single molecule detection of adenine has been shown using the disclosed methods and apparatus.

Rolling Circle Amplification

One picomole (pmol) of a rolling circle amplification (RCA) primer was added to 0.1 pmol of circular, single-stranded M13 DNA template. The mixture was incubated with 1×T7 polymerase 160 buffer (20 mM (millimolar) Tris-HCl, pH 7.5, 10 mM MgCl₂, 1 mM dithiothreitol), 0.5 mM dNTPs and 2.5 units of T7 DNA polymerase for 2 hours at 37° C., resulting in formation of an RCA product. A negative control was prepared by mixing and incubating the same reagents without the DNA polymerase.

SERS Detection of RCA Product

One μL of the RCA product and 1 μL of the negative control sample were separately spotted on an aluminum tray and air-dried. Each spot was rinsed with 5 μL of 1×PBS (phosphate buffered saline). The rinse was repeated three times and the aluminum tray was air-dried after the final rinse.

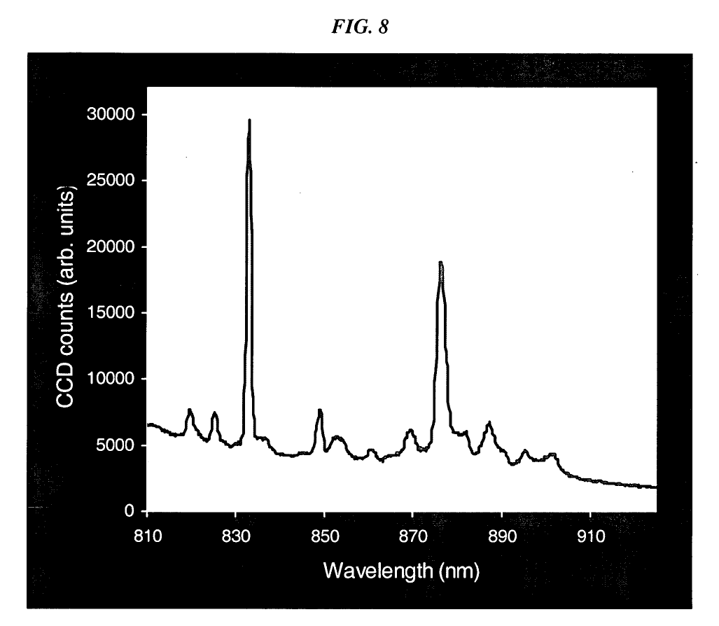

One milliliter of silver colloid solution prepared as above was diluted with 2 mL of distilled water. Eight microliters of the diluted silver colloid solution was mixed with 2 μL of 0.5 M LiCl and added to the RCA product spot on the aluminum tray. The same solution was added to the negative control spot. The Raman signals were collected as disclosed above. As demonstrated in FIG. 8, an RCA product was detectable by SERS, with emission peaks at about 833 and 877 nm. Under the conditions of this protocol, with an LiCl enhancer, the signal strength from the adenine moieties is stronger than those for guanine, cytosine and thymine. The negative control (not shown) showed that the Raman signal was specific for the RCA product, as no signal was observed in the absence of amplification.

### Example 5

### SERS Spectra of Labeled Nucleic Acids with Base Analogs

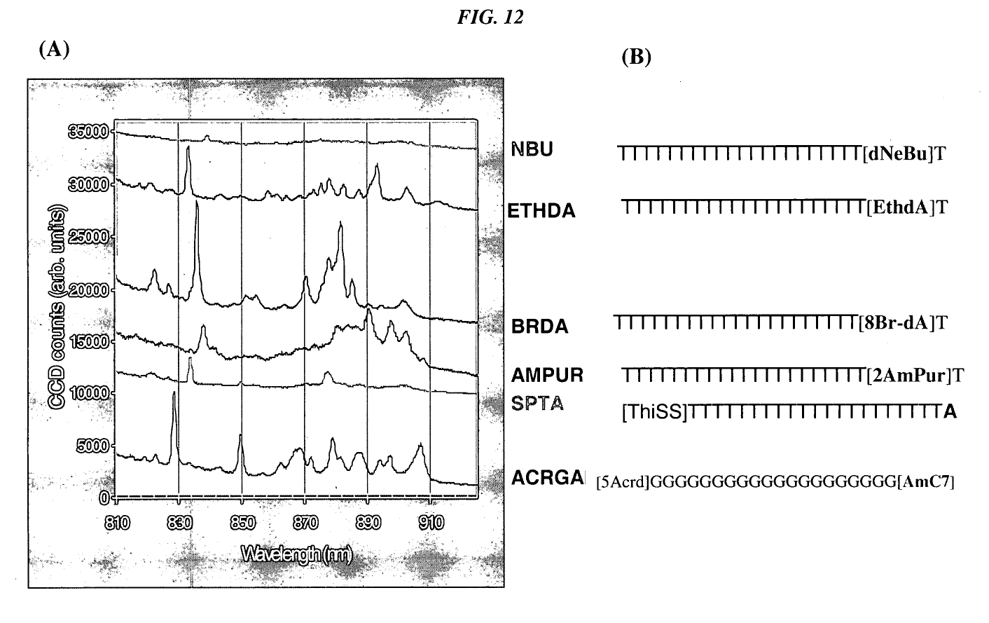

FIG. 12(A) shows the SERS spectra of several labeled nucleic acids, whose structures are shown in FIG. 12(B). A 10 μM solution of the indicated nucleic acids in LiCl was analyzed by surface enhanced Raman spectroscopy, as disclosed in Example 2. A 1 millisecond collection time was used. Other conditions were as disclosed in Example 2. The labeled nucleotides were custom synthesized by Qiagen-Operon (Alameda, Calif.). The Raman signals were generated by the following Raman labels, covalently incorporated into the indicated nucleic acids as shown in FIG. 12(B): NBU (5′-(T)20-deoxyNebularine-T-3); ETHDA (5′-(T)20-(N-ethyldeoxyadenosine)-T-3′); BRDA (5′-(T)20-(8-Bromoadenosine)-T-3′); AMPUR (5′-(T)20-(2-Aminopurine)-T-3′); SPTA (5′-ThiSS-(T)20-A-3); and ACRGAM (5′-acrydite-(G)20-Amino-C7-3′). As shown in FIG. 12(A), each Raman label produces a distinguishable SERS spectrum when incorporated into an oligonucleotide or nucleic acid.

All of the METHODS and APPARATUS disclosed and claimed herein can be made and executed without undue experimentation in light of the present disclosure. It will be apparent to those of skill in the art that variations may be applied to the METHODS and APPARATUS described herein without departing from the concept, spirit and scope of the claimed subject matter. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the claimed subject matter.

1. A method comprising:
a) synthesizing one or more labeled nucleic acids;
b) passing the labeled nucleic acids through nanopores;
c) detecting labeled nucleotides in the nucleic acids;
d) compiling a nucleotide distance map for each type of labeled nucleotide; and
e) characterizing the nucleic acid from the nucleotide distance maps.

2. The method of claim 1, wherein said characterization comprises identifying and/or sequencing the nucleic acid.

3. The method of claim 1, wherein each nanopore is operably coupled to a detector.

4. The method of claim 1, wherein only one labeled nucleic acid passes through a nanopore at a time.

5. The method of claim 1, wherein the length of time between passage of a first-labeled nucleotide through the nanopore and passage of a second labeled nucleotide through the nanopore corresponds to the distance along the labeled nucleic acid between the first and second nucleotides.

6. The method of claim 1, wherein labeled nucleotides are detected by electrical detection or photodetection.

7. The method of claim 1, wherein the labels are selected from the group consisting of photolabels, fluorescent labels, phosphorescent labels, chemiphotolabels, conductive labels, nuclear magnetic resonance labels, mass spectroscopy labels, electron spin resonance labels, electron paramagnetic resonance labels and Raman labels.

8. The method of claim 1, wherein at least one end of the labeled nucleic acid strand is attached to an identifiable label.

9. The method of claim 1, further comprising analyzing multiple copies of the same nucleic acid.

10. The method of claim 9, wherein each copy is labeled on a different set of nucleotide residues.

11. The method of claim 1, wherein each type of nucleotide is labeled.

12. The method of claim 11, wherein nucleic acids labeled on different types of nucleotides are separately analyzed.

13. The method of claim 1, wherein only one type of nucleotide is labeled.

14. The method of claim 1, wherein only purines or only pyrimidines are labeled.

15. The method of claim 14, further comprising analyzing both strands of a double-stranded nucleic acid.

16. An apparatus comprising:
a) at least one sub-device, each sub-device comprising an first chamber and a second chamber, said first and second chambers separated by sensor layers, the first and second chambers of each sub-device in fluid communication through one or more nanopores; and
b) one or more detectors operably coupled to the nanopores.

17. The apparatus of claim 16, wherein a single detector is capable of separately detecting signals from all sub-devices in the apparatus.

18. The apparatus of claim 16, further comprising an electrode in each first and second chamber, said electrodes operably coupled to a voltage regulator.

19. The apparatus of claim 16, further comprising a computer operably coupled to the one or more detectors.

20. The apparatus of claim 16, wherein the one or more detectors are Raman detectors.

21. The apparatus of claim 16, wherein said detector is selected from the group consisting of a photodetector, an electrical detector, an impedance detector and a voltage detector.

22. The apparatus of claim 16, wherein said sensor layers comprise a support layer, one or more photon sensing layers and two or more light opaque layers.

23. The apparatus of claim 16, wherein said sensor layers comprise at least one conducting layer and at least two insulating layers.

24. The apparatus of claim 23, wherein said conducting layer is operably coupled to an electrical detector.

25. The apparatus of claim 16, further comprising four sub-devices.

26. The apparatus of claim 16, wherein said nanopore is part of a nanotube or nanochannel.

27. A method comprising:
a) synthesizing one or more labeled nucleic acids;
b) passing the labeled nucleic acids through nanopores;
c) detecting labeled nucleotides in the nucleic acids by Raman spectroscopy;
d) compiling a nucleotide distance map for each type of labeled nucleotide; and
e) characterizing the nucleic acid from the nucleotide distance maps.

28. The method of claim 27, wherein the Raman spectroscopy is selected from the group consisting of surface enhanced Raman spectroscopy (SERS), surface enhanced resonance Raman spectroscopy (SERRS) and coherent anti-Stokes Raman spectroscopy (CARS).

29. The method of claim 26, wherein each of the four types of nucleotides is labeled.

30. The method of claim 29, wherein nucleic acids containing different types of labeled nucleotides are separately analyzed.
